SEQUENCE LISTING (9 sequences)

SEQ ID NO: 1 - RNA, 20 nt, Homo sapiens
agugguuuuaccuaugguag

SEQ ID NO: 2 - RNA, 22 nt, Homo sapiens
cagugguuuuacccuaugguag

SEQ ID NO: 3 - RNA, 21 nt, Homo sapiens
uaccacaggguagaaccacgg

SEQ ID NO: 4 - RNA, 100 nt, Homo sapiens
ugugucucucucuguguccugccagugguuuuacccuaugguagguuacgucaugcuguucuaccacaggguagaaccacggacaggauaccggggcacc

SEQ ID NO: 5 - RNA, 7 nt, Homo sapiens
gugguuu

SEQ ID NO: 6 - DNA, 14 bp, Artificial Sequence
  siRNA
cagggacccaggag

SEQ ID NO: 7 - RNA, 22 nt, Artificial Sequence
  siRNA
accacaggguagaaccacggac

SEQ ID NO: 8 - DNA, 15 bp, Artificial Sequence
  locked nucleic acids
tagggtaaaaccact

SEQ ID NO: 9 - DNA, 19 bp, Artificial sequence
  locked nucleic acid
cgtggttctaccctgtggt

(19) United States
(12) Patent Application Publication
(10) Pub. No.: US 2016/0024597 A1
(43) Pub. Date: Jan. 28, 2016

(54) miRNAs AS THERAPEUTIC TARGETS IN CANCER

(71) Applicant: The Research Foundation For The State University Of New York, Albany, NY (US)

(72) Inventors: Jingfang Ju, East Setauket, NY (US); Yuan Wang, Selden, NY (US); Bo Song, Port Jefferson, NY (US)

(21) Appl. No.: 14/879,700

(22) Filed: Oct. 9, 2015

Related U.S. Application Data
(63) Continuation of application No. 13/257,836, filed on Dec. 5, 2011.
(60) Provisional application No. 61/162,149, filed on Mar. 20, 2009.

(51) Int. Cl. C12Q 1/68

(52) U.S. Cl. CPC C12Q 1/6886; C12Q 2600/106; C12Q 2600/178; C12Q 2600/158

(57) ABSTRACT

Methods for modulating expression of a component of a cell, comprising contacting the cell with a nucleic acid comprising an miR-140 nucleic acid sequence in an amount sufficient to modulate the cellular component are provided. Overexpression of miR-140 inhibits cell proliferation in both U-2 OS (wt-p53) and HCT 116 (wt-p53) cell lines. Cells transfected with miR-140 are more resistant to chemotherapeutic agent methotrexate. mi-140 expression is related to HDAC4 protein expression. The claimed methods reduce the protein expression level of HDAC4 without degrading the target mRNA.

### CROSS-REFERENCE TO RELATED APPLICATIONS

This application is a continuation of U.S. application Ser. No. 13/257,836 filed Dec. 5, 2011, which is a 371 PCT/US2010/028191 filed Mar. 22, 2010 which claims the benefit of priority to U.S. Application No. 61/162,149, filed Mar. 20, 2009, all of which are incorporated herein by reference in their entireties.

### FIELD OF THE INVENTION

The present invention relates to characterization of miR-140 and related biological pathways, as well as the use of microRNAs (miRNAs) and other inhibitory polynucleotides for therapeutic, prognostic, and diagnostic applications.

### BACKGROUND OF THE INVENTION

miRNAs are small, non-coding single-stranded RNAs with predicted potential to regulate over 30% of the human protein coding genes at the post-transcriptional level, mainly by binding to the 3′-UTR of their mRNA targets as reported in, for example, Bartel D P, MicroRNAs: genomics, biogenesis, mechanism, and function. Cell. 2004; 116: 281-297; Lewis B P et al. Conserved seed pairing, often flanked by adenosines, indicates that thousands of human genes are microRNA targets. Cell. 2005; 120: 15-20; and Verghese E T et al., Small is beautiful: microRNAs and breast cancer—where are we now? J Pathol. 2008; 215: 214-221. Numerous studies in recent years have shown that miRNAs play important roles in multiple biological processes, such as development and differentiation, cell proliferation, apoptosis, metabolism, and stress response as reported in, for example, Yu Z R et al., Acyclin D1/microRNA 17/20 regulatory feedback loop in control of breast cancer cell proliferation. J Cell Biol. 2008; 182:509-517; Meng F Y et al., Involvement of human micro-RNA in growth and response to chemotherapy in human cholangiocarcinoma cell lines. Gastroenterology. 2006; 130: 2113-2129; Alvarez-Garcia I et al., MicroRNA functions in animal development and human disease. Development. 2005; 132: 4653-4662; Cheng A M et al., Antisense inhibition of human miRNAs and indications for an involvement of miRNA in cell growth and apoptosis. Nucleic Acids Res. 2005; 33: 1290-1297; and Raver-Shapira N et al., Transcriptional activation of miR-34a contributes to p53-mediated apoptosis. Mol Cell. 2007; 26: 731-743.

As an example, miR-34a has been found to be expressed in a p53-dependent manner and mediate some important functions of p53 activation, such as apoptosis, cell cycle arrest and senescence as reported in, for example, Chang T. C. et al., Transactivation of miR-34a by p53 broadly influences gene expression and promotes apoptosis. Mol. Cell 2007; 26: 745-752; He L. et al., A microRNA component of the p53 tumour suppressor network. Nature. 2007; 447: 1130-1134; and Raver-Shapira N. et al., Transcriptional activation of miR-34a contributes to p53-mediated apoptosis. Mol. Cell 2007; 26: 731-743. This effectively confirmed a number of miRNAs were involved in the p53 tumor suppressor gene suggested first by the inventors (See Xi Y. et al., Differentially regulated micro-RNAs and actively translated messenger RNA transcripts by tumor suppressor p53 in colon cancer. Clin Cancer Res. 2006; 12: 2014-2024). miR-143 and miR-145 were reported to display reduced level in the adenomatous and cancer stages of colorectal neoplasia (Michael M Z et al., Reduced accumulation of specific microRNAs in colorectal neoplasia. Mol Cancer Res. 2003; 1: 882-891). A recent report showed that miR-192 inhibited cell proliferation significantly in the colon cancer cell lines with wt-p53 status, further underscore the importance of miRNAs in modulating cell proliferation through p53 (See Bo Song et al., miR-192 regulates dihydrofolate reductase and cellular proliferation through the p53-miRNA circuit. Clin Cancer Res. 2008 in press).

Other cellular components, such as histone deacetylases (HDACs), mediate changes in nucleosome conformation and are important in the regulation of gene expression. Finnin, M. S., et al (1999). Structures of a histone deacetylase homologue bound to the TSA and SAHA inhibitors. Nature 401: 188-93. HDACs are involved in cell-cycle progression and differentiation, and their deregulation is associated with several cancers. Yang X J, Grégoire S. (2005). Class II histone deacetylases: from sequence to function, regulation, and clinical implication. Mol Cell Biol. 25: 2873-2874. Histone acetylation is important for regulating DNA chromatin structure and transcriptional control. Eberharter A, Becker, P B. (2002). Histone acetylation: a switch between repressive and permissive chromatin. Second in review series on chromatin dynamics. EMBO Rep 3: 224-229; Grozinger C, Schreiber, S L. (2002). Deacetylase enzymes: biological functions and the use of small-molecule inhibitors. Chem Biol. 9: 3-16; and Sengupta N, Seto, E. (2004). Regulation of histone deacetylase activities. J Cell Biochem 93: 57-67. HDAC isozyme can be categorized into three classes and HDAC4 belongs to class II, which can be regulated and shuttled between the cytoplasm and the nucleus in response to various signal transduction stimuli. In addition, class II HDACs exert their transcriptional co-repressor functions by interaction with other co-repressors or direct binding to (and sequestering) sequence-specific transcriptional factors such as MEF2, Runx3, and nuclear factor κB (NF-κB). Grozinger (2002); and Yang (2005).

There exists a need for better prognostic and diagnostic measures, treatment and control of neoplasm through application of small molecules to target cells to affect various cellular components, such as HDAC4, p53, and p21, involved directly or indirectly in regulation of cellular proliferation and neoplasia.

### SUMMARY OF THE INVENTION

In one embodiment, the invention provides a method of increasing proliferation of a cell, comprising contacting the cell with an inhibitory nucleic acid complementary to at least a portion of miR-140, in an amount effective to increase proliferation of the cell. In an embodiment, the nucleic acid is an antisense nucleic acid. In another embodiment, the nucleic acid is an siRNA, shRNA or an anti-miRNA. In another embodiment, the nucleic acid comprises a locked nucleic acid (LNA). In another embodiment, the cell is a cancer stem cell. In another embodiment, the cell is a neoplastic cell. In another embodiment, the nucleic acid is transfected.

The invention further provides a method of increasing the sensitivity of a cell to a chemotherapeutic agent, comprising contacting the cell with an inhibitory nucleic acid complementary to miR-140, in an amount effective to sensitize the cell to the chemotherapeutic agent. In an embodiment, the nucleic acid is an antisense nucleic acid. In another embodiment, the nucleic acid is an siRNA, shRNA or an anti-miRNA. In another embodiment, the nucleic acid comprises a locked nucleic acid (LNA). In another embodiment, the cell is a cancer stem cell. In another embodiment, the cell is a neoplastic cell. In another embodiment, the nucleic acid is transfected. In another embodiment, the chemotherapeutic agent is selected from methotrexate, doxorubicin, cisplatin, and ifosfamide

The invention further provides a method of increasing the sensitivity of a cell to radiation, comprising contacting the cell with an inhibitory nucleic acid complementary to at least a portion of miR-140, in an amount effective to sensitize the cell to radiation. In an embodiment, the nucleic acid is an antisense nucleic acid. In another embodiment, the nucleic acid is an siRNA, shRNA or an anti-miRNA. In another embodiment, the nucleic acid comprises a locked nucleic acid (LNA). In another embodiment, the cell is a cancer stem cell. In another embodiment, the cell is a neoplastic cell.

The invention further provides a method of treating a neoplasm in a subject, comprising administering to the subject an effective amount of a nucleic acid molecule that inhibits miR-140. In an embodiment, the method further comprises administering a second therapy, wherein inhibition of miR-140 sensitizes the neoplasm to the second therapy. In another embodiment, the second therapy comprises administering a chemotherapeutic agent. In another embodiment, the chemotherapeutic agent is selected from methotrexate, doxorubicin, cisplatin, and ifosfamide. In another embodiment, the second therapy comprises administering radiation to the subject. In another embodiment, the neoplasm is cancer. In yet another embodiment, the cancer is selected from the group consisting of colon cancer, pancreatic cancer, lung cancer, breast cancer cervical cancer, gastric cancer, kidney cancer, leukemia, liver cancer, lymphoma, ovarian cancer, prostate cancer, rectal cancer, sarcoma, skin cancer, testicular cancer, uterine cancer.

The invention further provides a method of diagnosing whether a neoplasm in a subject is resistant to chemotherapy comprising determining the level of expression of at least one of miR-140 and HDAC4 in cells of the neoplasm and identifying the neoplasm as chemotherapy resistant if the expression level of miR-140 is greater in the cells and/or the expression level of HDAC4 is less in the cells than in a control.

The invention further provides a method of determining whether a neoplasm comprises cells resistant to chemotherapy comprising determining the level of expression of at least one of miR-140 and HDAC4 in cells of the neoplasm and identifying the neoplasm as chemotherapy resistant if the expression level of miR-140 is greater in the cells and/or the expression level of HDAC4 is less in the cells than in a control. In an embodiment, the cells are stem-like cells. In another embodiment, the control is bulk neoplastic cells.

The invention further provides a kit for analysis of a pathological sample, the kit comprising in a suitable container RNA hybridization or amplification reagent for determining the level of miR-140. In an embodiment, the RNA hybridization reagent comprises a hybridization probe. In another embodiment, the RNA hybridization reagent comprises amplification primers.

The invention further provides a method of identifying an agent that promotes cell proliferation and sensitivity to chemotherapy agents. The method comprises contacting a cell that expresses miR-140 RNA with an agent; and comparing the level of miR-140 RNA in the cell contacted by the agent with the level of miR-140 RNA in a cell not contacted by the agent, wherein the agent is an inhibitor of the expression of miR-140 RNA if the expression of miR-140 RNA is reduced in the cell contacted by the agent. In an embodiment, the cell contacted by the agent overexpresses the miR-140 RNA.

### BRIEF DESCRIPTION OF THE FIGURES

FIG. 1A shows a sequence comparison analysis of 3′-UTRs of mouse and human HDAC4 mRNAs with miR-140 interaction site. FIG. 1B shows miRNA expression analysis of U-2 OS cells (wt-p53), MG63 cells (mut-p53), HCT 116 (wt-p53) and HCT 116 (null-p53) transfected with miR-140 or miR control by real time PCR expression analysis. FIG. 1C (a, b) shows mRNA expression of HDAC4 mRNA in U-2 OS cells (FIG. 1C(a)) and in HCT 116 (wt-p53) (FIG. 1C(b)) by real time qRT-PCR analysis. GAPDH was used as internal standard for normalization. FIG. 1D shows protein expression of HDAC4 in U-2 OS cells and in HCT 116 (wt-p53) analyzed by Western immunoblot, α-tubulin was used as a protein loading control. (*, p<0.05; **, p<0.01; n=3).

FIGS. 2A-2D show the impact of miR-140 on cell proliferation using WST-1 assay in U-2 OS cells (wt-p53) (FIG. 2A); HCT 116 (wt-p53) cells (FIG. 2B); MG63 cells (mut-p53) (FIG. 2C); and HCT 116 (null-p53) cells (FIG. 2D). Each cell group was transfected with 100 nM miR control or miR-140; cell numbers were determined by the WST-1 assay (n=6).

FIGS. 3A and 3B depict a cell cycle analysis by flow cytometry in U-2 OS cells (wt-p53) and MG63 cells (mut-p53) (FIG. 3A) or HCT 116 (wt-p53) cells and HCT 116 (null-p53) cells (FIG. 3B) transfected with 100 nM miR control or miR-140.

FIG. 4 depicts a western immunoblot analysis of p53, p21 expression in U-2 OS cells (wt-p53) and HCT 116 (wt-p53), α-tubulin was used as a protein loading control.

FIG. 5A depicts a chemosensitivity assay in HCT 116 (wt-p53) cells (A). Cells were transfected with 100 nM miR control, miR-140 or siHDAC4, cells were then treated with methotrexate for 72 hrs. Cell viability was determined by the WST-1 assay (n=6). CD133^{hi}/CD44^{hi} HCT 116 (wt-p53) colon cancer stem cells were sorted by FACS as shown in FIG. 5B. FIG. 5C shows expression level of miR-140 in colorectal cancer stem cells and normal cancer cells as determined by real time qRT-PCR analysis (*, p<0.05, n=3).

FIG. 6 shows miR-140 expression in colorectal cancer and normal colon mucosa specimens by real time qRT-PCR analysis. Relative gene expression values were calculated using samples with the highest expression level of miRNA as 100%. (p=0.04; Wilcoxon test).

FIG. 7 depicts a chemosensitivity assay in HCT 116 (wt-p53) cells. Cells were transfected with 100 nM miR-140, miR control or siHDAC4, and then treated with 5-fluorouracil (5-FU) for 72 h, and cell viability was determined by the WST-1 assay. miR control was used as the negative control. Numbers are indicated as mean±s.d.

FIG. 8 depicts a chemosensitivity assay in FACS-sorted CD133^{+hi}/CD44^{+hi} colon cancer stem-like cells. CD133^{+hi}/CD44^{+hi} colon cancer stem-like cells and control HCT 116 (wt-p53) cells were incubated with lethal dose of 5-FU (100 μM) for 48 h; the dead cells were determined by the fluorescein isothiocyanate (FITC) Annexin V and PI detection kit (top, **P<0.01, Student's t-test, n=3). CD133^{+hi}/CD44^{+hi} HCT 116 (wt-p53) colon cancer stem-like cells transfected with LNA anti-miR-140 became sensitive to 5-FU treatment. CD133^{+hi}/CD44^{+hi} cells were transfected with 100 nM of LNA anti-miR-140, 24 h later, cells were incubated with 100 μM of 5-FU for 48 h. The dead cells were determined by the FITC Annexin V and PI detection kit (lower panel, *P<0.05, Student's t-test, n=3).

FIG. 9 shows that Histone deacetylase 4 (HDAC4) is the target of miR-140. HCT 116 (wt-p53) and HCT 116 (null-p53) cells were transfected with LNA anti-miR-140 and scramble-miR (LNA-control), and HDAC4 protein was quantified by western immunoblot.

### DETAILED DESCRIPTION OF THE INVENTION

The inventors have discovered that miR-140 participates in regulation of cell proliferation. Further, the level of expression of miR-140 in cell or tissue affects sensitivity to chemotherapeutic agents and predicts the effectiveness of chemotherapy. In particular, high levels of miR-140 reduce proliferation and increase resistance to chemotherapeutic agents, while low levels of miR-140 promote proliferation and sensitivity to chemotherapeutic agents. Also, miR-140 binds to HDAC-4 and reduces the protein expression level of HDAC4 without degrading the target mRNA. Overexpression of miR-140 inhibits cell proliferation in both U-2 OS (wt-p53) and HCT 116 (wt-p53) cell lines, but with less impact in MG63 (mut-p53) and HCT 116 (null-p53) cells. The inventors have found that miR-140 induces both G1 and G2 arrest only in U-2 OS (wt-p53) cells and HCT 116 (wt-p53) cells. In this regard, p53 and p21 were significantly induced by miR-140 only in cell lines containing wild type p53. Moreover, cells transfected with miR-140 were more resistant to chemotherapeutic agent methotrexate. The expression of endogenous miR-140 is highly elevated in CD133^{+hi}CD44^{+hi} colon cancer stem cells compared to control colon cancer cells, indicating that slow proliferating tumor stem cells may be avoiding damage caused by chemotherapeutic agents mediated, in part, by miR-140. Thus, miR-140 is a candidate target to develop novel therapeutic strategy to overcome drug resistance.

Human miR-140 (5′-agugguuuua ccuaugguag-3′, SEQ ID NO:1; 5′-cagugguuuuacccuaugguag-3′, hsa-miR-140-5p, SEQ ID NO:2) is encoded by a gene located on human chromosome 16 (GenBank Accession NT_{—}010498). miR-140 is located within a larger sequence that forms a stem-loop structure, and which further includes a second miRNA (5′-uaccacaggguagaaccacgg-3′, hsa-miR-140-3p, SEQ ID NO:3). The sequence 5′-ugugucucucucuguguccugccagugguuuuacccuaugguagguuacgucaugcuguucuaccacaggguagaa ccacggacaggauaccggggcacc-3′ (SEQ ID NO:4) includes bases upstream and downstream of miR-140 (hsa-miR-140-5p and hsa-miR-140-3p are underlined). (See Sanger miRBase Accession MI0000456).

In certain aspects, the invention is directed to methods for the assessment, analysis, and/or therapy of a cell or subject where certain genes have a reduced or increased expression (relative to normal) as a result of an increased or decreased expression of miR-140. The expression profile and/or response to miR-140 expression or inhibition may be indicative of a disease or an individual with a pathological condition such as, for example, cancer.

According to the invention, inhibitors of miRNA-140 include antisense nucleic acids and other inhibitory nucleic acids or molecules. Antisense nucleic acids are effective in inhibiting human miRNAs. Antisense nucleic acids include non-enzymatic nucleic acid compounds that bind to a target nucleic acid by, for example, RNA-RNA, RNA-DNA, DNA-PNA or PNA-PNA interactions and effect the target nucleic acid. Generally, these molecules are complementary to a target sequence along a single contiguous sequence of the antisense nucleic acid. In this embodiment, the antisense nucleic acid inhibits miR-140.

In another embodiment, an antisense nucleic acid or other inhibitory nucleic acid binds to a substrate nucleic acid and forms a loop. In this embodiment, the antisense nucleic acids may be complementary to two or more non-contiguous substrate sequences and/or two or more non-contiguous sequence portions of an antisense nucleic acid may be complementary to a target sequence.

In another embodiment, an antisense nucleic acid is complementary to a guide strand of an miRNA positioned in the RNA silencing complex. In another embodiment, antisense nucleic acids may be used to target a nucleic acid by means of DNA-RNA interactions. In this embodiment, RNase H is activated to digest the target nucleic acid as would be understood by one of ordinary skill in the art. For example, the antisense nucleic acids may comprise one or more RNAse H activating region capable of activating RNAse H to cleave a target nucleic acid. The RNase H activating region may comprise any suitable backbone. For example, in this embodiment, the RNase H activating region may comprise a phosphodiester, phosphorothioate, phosphorodithioate, 5′-thiophosphate, phosphoramidate and/or methylphosphonate.

Generally, inhibitory nucleic acids are polynucleotides or polynucleotide analogs that are complimentary to a portion of a target gene (e.g., miR-140) and reduce or prevent expression of the target gene product (e.g., mRNA or protein) Inhibitory polynucleotides are typically greater than 10 bases or base pairs in length and are composed of ribonucleotides and/or deoxynucleotides or a modified form of either type of nucleotide, and may be single and/or double stranded. For example, inhibitory nucleic acids may comprise phosphorothioate-type oligodeoxyribonucleotides, phosphorodithioate-type oligodeoxyribonucleotides, methylphosphonate-type oligodeoxyribonucleotides, phosphoramidate-type oligodeoxyribonucleotides, H-phosphonate-type oligodeoxyribonucleotides, triester-type oligodeoxyribonucleotides, alpha-anomer-type oligodeoxyribonucleotides, peptide nucleic acids, locked nucleic acids, and nucleic acid-modified compounds. It will be readily apparent to one of ordinary skill in the art that other oligonucleotides are within the scope and spirit of this invention.

Inhibitory nucleic acid may be based on 2′-modified oligonucleotides containing oligodeoxynucleotide gaps with internucleotide linkages modified to phosphorothioates for nuclease resistance. The presence of methylphosphonate modifications increases the affinity of the oligonucleotide for its target RNA and thus increases its effectiveness in inhibiting the target RNA. This modification also increases the nuclease resistance of the modified oligonucleotide.

Inhibitory nucleic acids may comprise a backbone modification. For example, oligomers having modified backbones may include those that retain a phosphorus atom in the backbone and those that do not have a phosphorus atom in the backbone. Nucleotides with modified backbones include, but are not limited to, phosphorothioates, chiral phosphorothioates, phosphorodithioates, phosphotriesters, aminoalkyl-phosphotriesters, methyl and other alkyl phosphonates, phosphinates, phosphoramidates, thionophosphoramidates, thionoalkylphosphonates, thionoalkylphosphotriesters, and boranophosphates. Other forms, including, but not limited to, salts, mixed salts and free acid forms, are also contemplated.

Oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include, but are not limited to, those having morpholino linkages, siloxane backbones, sulfide, sulfoxide and sulfone backbones, formacetyl and thioformacetyl backbones, methylene formacetyl and thioformacetyl backbones, alkene containing backbones, sulfamate backbones, methyleneimino and methylenehydrazino backbones, sulfonate and sulfonamide backbones, and/or amide backbones. Further, the oligomers may include nucleotides with substituents that bias or lock the conformation of the backbone, such as, for example, “locked” nucleotides.

Locked nucleic acid (LNA) nucleosides are a class of nucleic acid analogues in which the ribose ring is “locked” by a methylene bridge connecting the 2′-O atom and the 4′-C atom. LNA nucleosides contain the common nucleobases (T, C, G, A, U and mC) and are able to form base pairs according to standard Watson-Crick base pairing rules. However, by “locking” the molecule with the methylene bridge the LNA is constrained in the ideal conformation for Watson-Crick binding. When incorporated into a DNA oligonucleotide, LNA therefore makes the pairing with a complementary nucleotide strand more rapid and increases the stability of the resulting duplex. Incorporation of LNA monomers into an oligonucleotide increases the duplex melting temperature (Tm) by 2-8° C. per LNA monomer. Thus, inhibitory nucleic acids containing LNA monomers are relatively short, typically 7-20mers, or 8-15mers.

Accordingly, the invention provides for the use of single stranded oligonucleotides having a length of between 8 and 17 nucleobase units, wherein at least one of the nucleobase units of the single stranded oligonucleotide is a high affinity nucleotide analogue, such as a Locked Nucleic Acid (LNA) nucleobase unit, and wherein the single stranded oligonucleotide is at complementary to a human miRNA sequence, such as miR-140. According to the invention, complementary means that base sequence of the oligonucleotide is at least 85% identical, or at least 90% identical, or at least 95% identical, or identical to the complement of miR-140 or a portion thereof. One oligonucleotide comprising LNA nucleobase units useful for inhibiting miR-140 has the sequence 5′-TAGGGTAAAACCACT (SEQ ID NO:7). Another has the sequence 5′-CGTGGTTCTACCCTGTGGT (SEQ ID NO:8). MicroRNA inhibitors, for example, polynucleotides containing locked nucleic acids, are commercially available.

In another embodiment, the modification may also comprise one or more substituted sugar moieties. For example, the RNase H activating region may comprise deoxyribose, arabino and/or fluoroarabino nucleotide sugar chemistry. Such modifications may also include 2′-O-methyl and 2′-methoxyethoxy modifications, 2′-dimethylaminooxyethoxy, 2′-aminopropoxy and 2′-fluoro, and modifications at other positions on the oligonucleotide or other nucleobase oligomer, particularly the 3′ position of the sugar on the 3′ terminal nucleotide. Nucleobase oligomers may also have sugar mimetics. In another embodiment, both the sugar and the internucleoside linkage may be replaced with novel groups. The nucleobase units are maintained for hybridization with a nucleic acid molecule of miR-140.

Morpholino oligomers are short chains of about 10 to about 30 morpholino subunits. Morpholinos may also be about 15 to about 25, or about 18 to about 22 subunits long. Each subunit is comprised of a nucleic acid base, a morpholine ring and a non-ionic phosphorodiamidate intersubunit linkage. Morpholinos do not degrade their RNA targets, but instead act via a steric blocking mechanism. Systemic delivery into cells in adult organisms can be accomplished by using covalent conjugates of Morpholino oligos with cell penetrating peptides. An octa-guanidinium dendrimer attached to the end of a Morpholino can deliver the modified oligonucleotide (called a Vivo-Morpholino) from the blood to the cytosol. (Moulton, J. D., Jiang S. (2009). Gene Knockdowns in Adult Animals: PPMOs and Vivo-Morpholinos. Molecules, 14 (3): 1304-23; Morcos, P. A., Li Y. F., Jiang S. (2008). Vivo-Morpholinos: A non-peptide transporter delivers Morpholinos into a wide array of mouse tissues. BioTechniques 45 (6):616-26).

According to another embodiment, the invention relates to the use of interference RNA (RNAi) to reduce expression of miR-140. RNAi comprise double stranded RNA that can specifically block expression of a target gene. Double-stranded RNA (dsRNA) blocks gene expression in a specific and post-transcriptional manner. RNAi provides a useful method of inhibiting gene expression in vitro or in vivo. RNAi can comprise either long stretches of dsRNA identical or substantially identical to the target nucleic acid sequence or short stretches of dsRNA identical to or substantially identical to only a region of the target nucleic acid sequence.

RNAi includes, but is not limited to, small interfering RNAs (siRNAs), small hairpin RNAs (shRNAs) and anti-miRNA, and other RNA species, such as non-enzymatic nucleic acids, which can be cleaved in vivo to form siRNAs. RNAi may also include RNAi expression vectors capable of giving rise to transcripts which form dsRNAs or shRNAs in cells, and/or transcripts which can produce siRNAs in vivo.

The inhibitory nucleic acid is complimentary or partially complimentary to the target gene mRNA. The complimentary or partially complimentary region of the target gene mRNA may be in the 5′ untranslated region (UTR), 3′ UTR, and/or in the coding region. siRNAs are double-stranded RNA molecules, typically about 19 to about 30 nucleotides in length, more preferably 19-23 or 21-23 nucleotides in length and having a 2 nucleotide overhang at the 3′ end of each strand. For example, an siRNA to repress targets of miR-140 consists of SEQ ID NO:5 and SEQ ID NO:6. Methods for designing specific siRNAs based on an mRNA sequence are well known in the art (see e.g., Brummelkamp, T. R. et al. (2002) A system for stable expression of short interfering RNAs in mammalian cells. Science 19, 550-553; Ui-Tei, K. et al. (2004) Guidelines for the selection of highly effective siRNA sequences for mammalian and chick RNA interference. Nucleic Acids Res. 32, 936-948; Hohjoh H. (2004) Enhancement of RNAi activity by improved siRNA duplexes. FEBS Lett. 557, 193-8; and Yuan, B., et al. siRNA Selection Server: an automated siRNA oligonucleotide prediction server. (2004) Nucleic Acids Res. 32, W130-134). In addition, design algorithms are available on the websites of many commercial vendors that synthesize siRNAs, including Ambion, Clontech, Dharmacon, GenScript, and Qiagen.

The siRNAs effectively recruit nuclease complexes and guide the complexes to the target mRNA by pairing to the specific sequences. As a result, the target mRNA is degraded by the nucleases in the protein complex. In certain embodiments, the 21-23 nucleotides siRNA molecules comprise a 3′ hydroxyl group. In certain embodiments, the siRNA can be generated by processing of longer double-stranded RNAs, for example, in the presence of the enzyme dicer. The siRNA molecules can be purified using a number of techniques known to those of skill in the art such as, for example, gel electrophoresis, non-denaturing column chromatography, chromatography, glycerol gradient centrifugation, and/or affinity purification with an antibody.

Small interfering RNAs can be expressed in the form of short, hairpin loop polynucleotides known as short hairpin RNAs (shRNAs) comprising the siRNA sequence of interest and a hairpin loop segment. Short hairpin RNAs are available through commercial vendors, which often provide online algorithms useful for designing shRNAs (e.g., Clontech, Invitrogen, ExpressOn, Gene Link, and BD Biosciences). shRNAs may be engineered in cells or in an animal to ensure continuous and stable suppression of a desired gene. It is recognized in the art that siRNAs can be produced by processing a shRNA in the cell. When expressed in a cell, shRNA is rapidly processed by intracellular machinery into siRNA. Expression of shRNAs may be accomplished by ligating the DNA sequence corresponding to the shRNA into an expression construct, for example the cloning site of a double-stranded RNA (dsRNA) expression vector. Expression may be driven by RNA polymerase III promoters. Expression vectors may be plasmid vectors including retrovirus, lentivirus, adenovirus, and adeno-associated virus based systems. Vectors for expression of shRNAs are commercially available from vendors such as Clontech, Invitrogen, Millipore, Gene Therapy Systems, Ambion and Stratagene. Methods for DNA and RNA manipulations, including ligation and purification, are well known to those skilled in the art (See e.g., Sambrook, J. and Russel, D. W., (2001) Molecular Cloning: A Laboratory Manual, Third Edition. Cold Spring Harbor Laboratory Press, Cold Spring Harbor, N.Y.; and Current Protocols in Molecular Biology, (2001) John Wiley & Sons, Inc.).

The RNA may be introduced in an amount which allows delivery of at least one copy per cell. Higher doses of double-stranded material may yield more effective inhibition, while lower doses may also be useful for specific applications Inhibition is sequence-specific in that nucleotide sequences corresponding to the duplex region of the RNA are targeted for genetic inhibition.

In one embodiment, the invention provides an inhibitory nucleic acid molecule (a polynucleotide) that is complementary to a portion of miR-140 (SEQ ID NOS:1-4) and is inhibitory to miR-140. In an embodiment of the invention, the inhibitory nucleic acid molecule is up to about 50 bases in length. In another embodiment of the invention, the inhibitory nucleic acid molecule is from about 8 to about is up to 30 bases in length. It is noted that the miR-140 precursor (SEQ ID NO:4) comprises a sequence capable of self-complementation to form a stem-loop structure. Thus, in some embodiments, nucleic acid molecules are complementary to both miR-140 and also to an miRNA target mRNA. Accordingly, they inhibit miR-140 are also the miR-140 target. In another embodiment of the invention, the inhibitory nucleic acid molecule is not complementary to a sequence that is a target of miR-140. For example, in one embodiment of the invention, the inhibitory nucleic acid molecule that inhibits miR-140 does not contain a subsequence that is complementary to an miR-140 binding site at the 3′-UTR of HDAC4 mRNA. Accordingly, HDAC4 activity is not reduced when miR-140 activity is reduced. In one such embodiment, the inhibitory nucleic acid molecule does not contain the nucleic acid sequence gugguuu (SEQ ID NO:5).

In an embodiment, the nucleic acid molecule is an antisense nucleic acid molecule. The antisense nucleic acid molecule includes a sequence having at least 85% sequence identity over its length to the complement of SEQ ID NO:1 and/or SEQ ID NO:2 and/or SEQ ID NO:3. As mentioned above, in certain embodiments, the antisense nucleic acid is selected to not be complementary to a sequence that is a target of miR-140. In another embodiment, an expression vector comprises the inhibitory nucleic acid molecule. The inhibitory nucleic acid may be operably linked to a promoter suitable for expression in a mammalian cell. The vector may be a viral vector. In another embodiment, a cell comprises the vector.

Sequence identity may be optimized by sequence comparison and alignment algorithms known in the art and calculating the percent difference between the nucleotide sequences. In this embodiment, the preferred sequence identity between the inhibitory RNA and the portion of the target gene is greater than 90%, 95%, 96%, 97%, 98%, 99% or 100%. Alternatively, the duplex region of the RNA may be defined functionally as a nucleotide sequence that is capable of hybridizing under specified conditions with a portion of the target gene transcript.

In this embodiment, anti-miRNA nucleic acids are nucleic acids designed to specifically bind to and inhibit endogenous miRNA molecules. It is recognized that anti-miRNA down-regulates the operation of miRNA in a cell.

In another embodiment, the invention relates to the use of suitable ribozyme molecules, such as, for example, RNA endoribonucleases and hammerhead ribozymes, designed to catalytically cleave mRNA transcripts to prevent translation of mRNA. Hammerhead ribozymes cleave mRNAs at locations dictated by flanking regions that form complementary base pairs with the target mRNA, which have a base sequence of 5′-UG-3′.

According to another embodiment, polynucleotide or expression vector therapy for treating neoplasia featuring a polynucleotide encoding an inhibitory nucleic acid molecule or analog thereof that targets miR-140 is provided. In this embodiment, the antisense nucleic acid may cause inhibition of expression by hybridizing with the miRNA and/or genomic sequences encoding the miRNA. Expression vectors encoding inhibitory nucleic acid molecules can be delivered to cells of a subject having a neoplasia in a form in which they can be taken up and expressed so that therapeutically effective levels may be achieved. The expression vector produces an oligonucleotide which is complementary to at least a unique portion of the target miRNA. Methods for delivery of the polynucleotides to the cell according to the invention include, but are not limited to, using a delivery system such as viral vectors, liposomes, polymers, microspheres, gene therapy vectors, and naked DNA vectors. Such nucleic acid probes may also be modified so that they are resistant to endogenous nucleases such as, for example, exonucleases and/or endonucleases, and are therefore stable in vivo.

Inhibitory nucleic acid molecule expression for use in polynucleotide therapy methods can be directed from any suitable promoter and regulated by any appropriate mammalian regulatory element. Promoters may include, but are not limited to, the human cytomegalovirus, simian virus 40, and/or metallothionein promoters. In this embodiment, enhancers known to preferentially direct gene expression in specific cell types can be used to direct the expression of a nucleic acid. The enhancers used can include, without limitation, those that are characterized as tissue- or cell-specific enhancers.

Non-exclusive examples of inhibitory polynucleotides are DNA and RNA.

Delivery of inhibitory polynucleotides may be local (i.e., to the site of the cell mass, affected tissue or neoplasm) or systemic (i.e., delivery to the circulatory or lymphatic systems). Local injection avoids many of the difficulties associated with intravenous administration, such as rapid elimination. In addition, helper molecules (for example, cationic lipids or polymers) or physical methods (for example electroporation, sonoporation, or hydrodynamic pressure) can be employed to facilitate intracellular entrance of the inhibitory polynucleotide. In addition, local production of inhibitory polynucleotides such as siRNA by genes encoding for shRNA can ensure prolonged levels of the dsRNA in the target cells.

The inhibitory polynucleotide may be targeted to the cell mass, affected tissue or neoplasm, or to particular cells in the cell mass, tissue, or neoplasm, by associating the inhibitory polynucleotide to a targeting molecule. The targeting molecule may be linked to the inhibitory polynucleotide by a covalent bond or may be associated ionically or by integration into the targeting mechanism (e.g., as part of the liposome, nanoparticle, or expressed on the surface of a donor cell). Targeting molecules include antibodies, and cell-penetrating peptides. Non-exclusive examples of antibodies are those that bind to antigens on the surface of the affected tissue or neoplasm. For example, antibodies that bind to CD133 or CD44 can be used for targeted delivery of mir-140 inhibitory polynucleotides to stem-like cells, including cancer stem cells. In addition, the inhibitory polynucleotide may be complexed with cationic lipids, cholesterol, peptides, polyethyleneimine, and/or condensing polymers or packaged in a liposome, nanoparticle, virus, bacteria, or in a donor cell. In one embodiment the donor cell is an immune privileged cell such as a MSC. (see, e.g., Xie, F. Y., et al. (2006). Harnessing in vivo siRNA delivery for drug discovery and therapeutic development. Drug Discovery Today, 11:67-73; Oliveira, S. et al. (2006) Targeted Delivery of siRNA. J. Biomed. Biotech. 2006:1-9; Whitehead, K. A., et al. (2009) Knocking Down Barriers; Advances in siRNA Delivery. Nature Reviews, 8:129-138).

Transducing viral vectors such as, for example, retroviral, adenoviral, lentiviral and adeno-associated viral vectors, can be used as expression vectors for somatic cell gene therapy. Viral vectors are especially useful because of their high efficiency of infection and stable integration and expression. In this embodiment, for example, a polynucleotide encoding an inhibitory nucleic acid molecule can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus.

In another embodiment, a non-viral approach may be employed for the introduction of an inhibitory nucleic acid molecule therapeutic to a cell of a patient diagnosed as having a neoplasia. For example, an inhibitory nucleic acid molecule that targets miRNA-140 can be introduced into a cell by administering the nucleic acid in the presence of lipofection, asialoorosomucoid-polylysine conjugation, or by micro-injection under surgical conditions. In this embodiment, the inhibitory nucleic acid molecules are administered in combination with a liposome and protamine. Gene transfer can also be achieved using non-viral means involving transfection in vitro. Such methods include the use of calcium phosphate, DEAE dextran, electroporation, and protoplast fusion. Liposomes can also be beneficial for delivery of DNA into a cell. According to the invention, the nucleic acid molecules that target miRNA-140 can be directed to specific cell types. For example, liposomes or other carriers can be targeted to cell surface antigens characteristic of a particular cell type. In an embodiment of the invention, the inhibitory nucleic acid molecules are targeted to an antigen characteristic of a cancer stem cells, including, but not limited to, CD133 and/or CD44.

For any particular subject, the specific dosage regimes should be adjusted over time according to the individual need and the professional judgment of the person administering or supervising the administration of the compositions.

Methods of modulating expression of cellular components in an amount sufficient to modulate the cellular component are also provided. In various embodiments, the cellular components to be modulated may comprise one or more of miR-140, p21, p53, HDAC4 or any cellular component regulated by these components. One of ordinary skill in the art would recognize that other cellular components may be modulated and are within the scope and spirit of this invention.

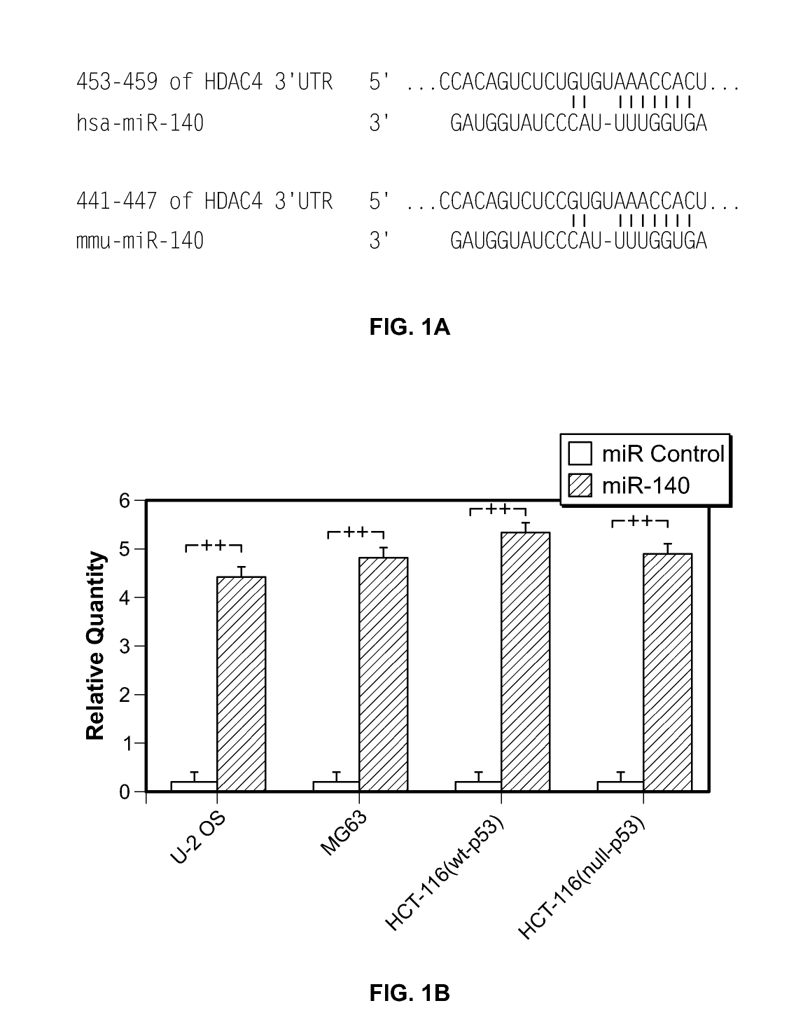

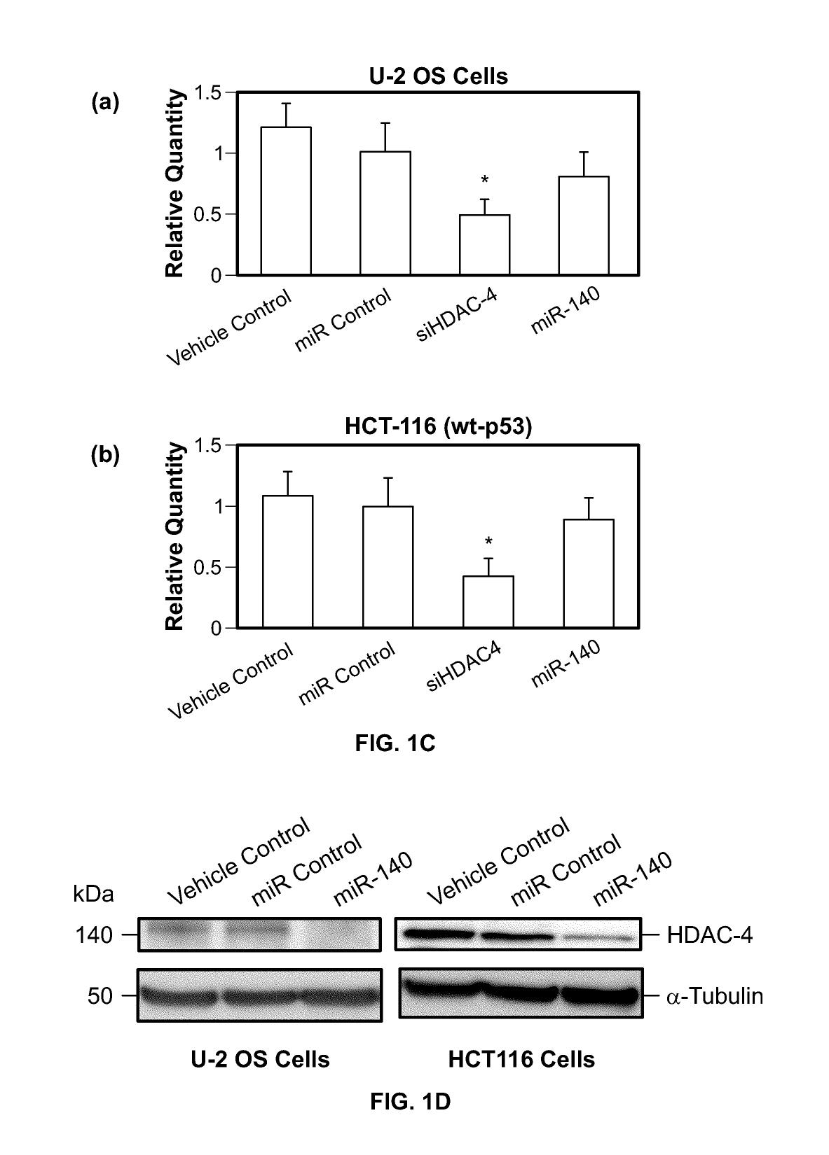

The inventors analyzed the human miR-140 sequence and confirmed that the sequence of the mouse mmu-miR-140 has the same sequence of human miR-140 and it is highly conserved (FIG. 1A). The 3′-UTR interaction site of the mouse HDAC4 with mouse miR-140 was also identical to the human HDAC4. They experimentally confirmed that one of the important targets of miR-140 is HDAC4. miR-140 reduced the expression level of HDAC4 protein without degradation of the target mRNA.

The inventors discovered that overexpression of miR-140 significantly inhibited cellular proliferation in cancer cell lines containing wild type p53. This was achieved, at least in part, by the induction of both G1 and G2 cell cycle arrest along with induction of p21. This effect, however, was largely absent in cell lines with either mutant or null p53. These results indicated that the impact of miR-140 on cell cycle control and cellular proliferation was, in part, dependant on the presence of functional wild type p53. Cells transfected with miR-140 were more resistant to chemotherapeutic agents such as methotrexate and 5-fluorouracil due to reduced proliferation. The expression of endogenous miR-140 was highly elevated in CD133^{+hi}CD44^{+hi} colon cancer stem cells compared to control colon cancer cells, suggesting that tumor stem cells may be avoiding cellular and DNA damage caused by chemotherapy with a reduced proliferating phenotype mediated, at least in part, by miR-140.

Furthermore, miR-140 expression level was decreased in clinical colorectal specimens compared to adjacent normal tissues of the same patients, suggesting the lowered levels of miR-140 in tumors are contributing the fast proliferating phenotype in differentiated non colon cancer stem cells. miR-140 is a candidate target to develop novel therapeutic strategy to overcome drug resistance.

The inventors have found that colon cancer stem cells depend, at least in part, on elevated levels of certain miRNAs, including miR-140, for their reduced cell proliferation phenotype. The advantage of tumor stem cells using miRNAs to achieve this is that translational control by an miRNA is an acute response, readily reversible without permanently degrading its target mRNAs such as HDAC4 or trigger apoptosis. This also suggests why half of the colon cancer cases containing wild type p53 are still resistant to chemotherapeutic treatment. This mechanism also provides a novel approach to killing colon cancer stem cells by inhibiting miR-140 and subsequently eliminating them with chemotherapeutic agents.

To investigate the direct impact of miR-140 on cellular proliferation and chemosensitivity, miR-140 was ectopically expressed using transient transfection in both osteosarcoma and colon cancer cell lines with different p53 status. The inventors discovered that that the impact of miR-140 on cellular proliferation was depended on, at least in part, the presence of wild type p53 tumor suppressor gene. Both G1 and G2 cell cycle arrest triggered by transient miR-140 overexpression was also largely depended on p53 and p21 induction. This is consistent with the finding that HDAC4 suppresses the expression of p21. For example, recent studies have shown that HDAC4 promotes growth of colon cancer cells via repression of p21. Wilson A J, Byun D S, Nasser S, Murray L B, Ayyanar K, Arango D et al (2008); and Mol Biol Cell 19: 4062-75. Wilson (2008). Thus, reduced expression of HDAC4 by miR-140 will release the suppressive control for p21 expression to allow cell cycle control.

These findings suggest that miR-140, either directly or indirectly mediated by p53, controls cell cycle and cell proliferation. p53 and p21, a downstream target of the p53 growth control pathway, are reported to block cells at G2 checkpoint mainly through inhibiting Cdc2 activity, the cyclin-dependent kinase that normally drives cells into mitosis, which is the ultimate target of pathways that mediate rapid arrest in G2 in response to DNA damage. See, e.g., Taylor, W. R. et al., 2001, Regulation of the G2/M transition by p53. Oncogene 20:1803-15; Stark, G. R. et al., 2006, Control of the G2/M transition. Mol. Biotechnol. 32:227-48; and Bunz, F. et al., 1998, Requirement for p53 and p21 to sustain G2 arrest after DNA damage. Science 282:1497-501.

The inventors have discovered that miR-140 can induce G2-arrest in HCT-116 (wt-p53) and U-2 OS cells. Transfection of precursors of these miRNAs into HCT-116 (wt-p53) and U-2 OS cells to indicate that over-expression of miR-140 led to a significant increase of the p53 and p21 protein in both HCT-116 (wt-p53) and U-2 OS cells. As exemplified herein, miR-140 contributes to the inhibition of cell proliferation at least partially by the induction of G2-arrest in HCT-116 (wt-p53) and U-2 OS cells, which was through over-expression of G2-checkpoint genes p53 and p21.

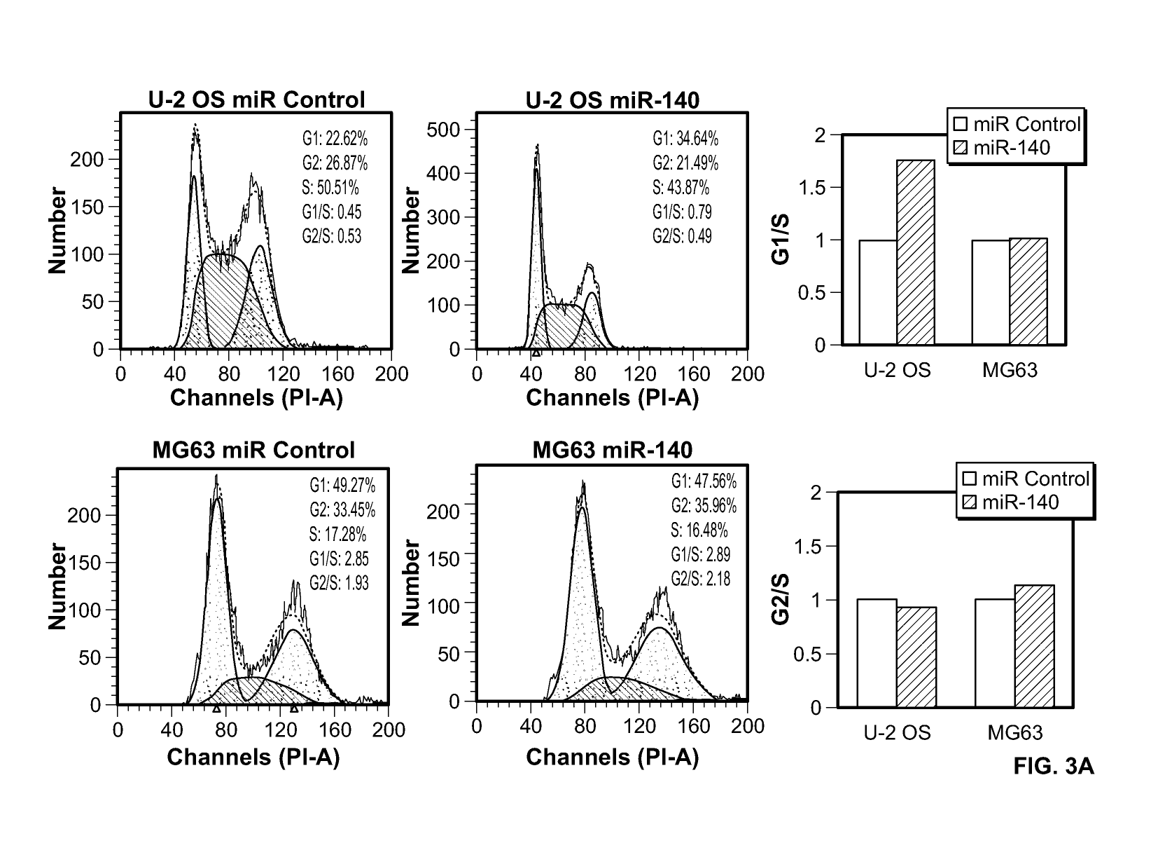

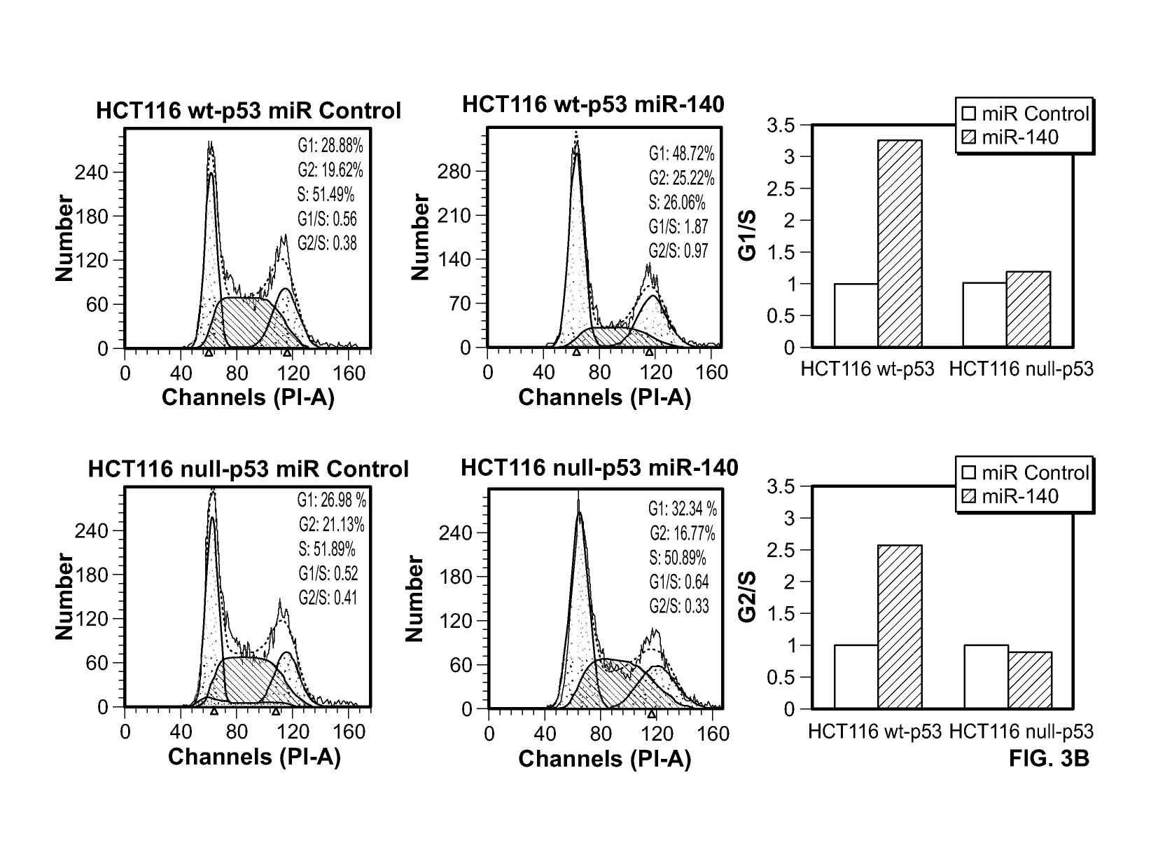

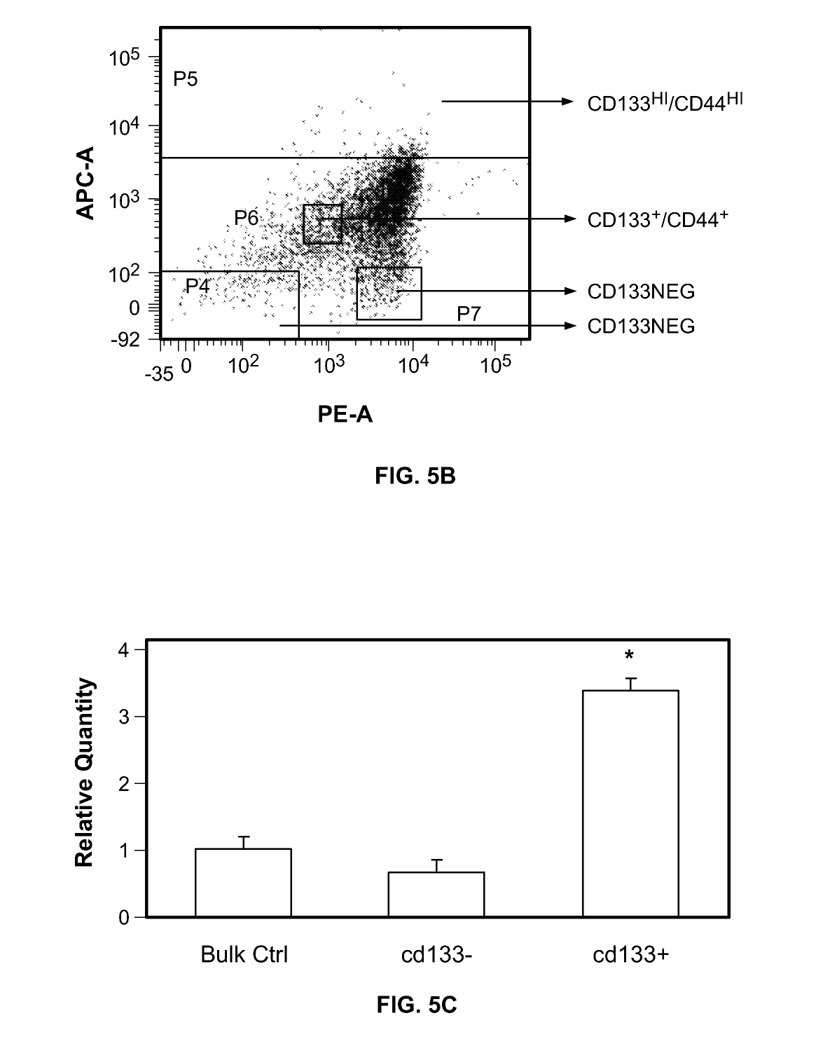

The inventors discovered that miR-140 suppresses cell proliferation. Despite the reduced levels of HDAC4, instead of sensitizing tumor cells to chemotherapeutic agents, ectopically overexpressing miR-140 causes more resistance to methotrexate treatment (FIG. 5) and 5-fluorouracil treatment (FIG. 7). While not binding this invention to any particular mechanism, this could be due to several possible reasons. One is that miR-140 regulates translational rate of many mRNA transcripts. The overall impact on genes and pathways are more important than a particular target. Another reason is that miR-140 reduces cell proliferation rate by decreasing S phase of the cell cycle and increased both G1 and G2 arrest (FIG. 3). In general, slowly proliferating or resting cells are more resistant to treatment with agents such as methotrexate and 5-fluorouracil that act during the S phase of the cell cycle to cause DNA damage. Elevated p21 may also contribute to such resistance to methotrexate. Bunz F, Hwang P M, Torrance C, Waldman T, Zhang Y, Dillehay L et al (1999). Disruption of p53 in human cancer cells alters the responses to therapeutic agents. J Clin Invest 104: 263-9.

Tumor cells are heterogeneous and bear a diversity of genetic changes. Cancer stem cells are cancer initiating cells, exhibit low rate of division and proliferation in their niche that help them to avoid chemotherapy and radiation. Zou G M (2008). Cancer initiating cells or cancer stem cells in the gastrointestinal tract and liver. J Cell Physiol 217: 598-604. This is the major difference between cancer stem cells and fast proliferating differentiated cancer cells which can be eliminated by chemotherapy treatment. With this in mind, the inventors analyzed the miR-140 expression levels from isolated CD133^{hi}/CD44^{hi} colon cancer stem cells using real time qRT-PCR. Both CD133 and CD44 have been reported to be important cell surface markers of colon cancer stem cells. Dalerba P, Dylla S J, Park I K, Liu R, Wang X, Cho R W et al (2007). Phenotypic characterization of human colorectal cancer stem cells. Proc Natl Acad Sci USA 104: 10158-63; Du L, Wang H, He L, Zhang J, Ni B, Wang X et al (2008). CD44 is of functional importance for colorectal cancer stem cells. Clin Cancer Res 14: 6751-60; O'Brien C A, Pollett A, Gallinger S, Dick J E (2007). A human colon cancer cell capable of initiating tumour growth in immunodeficient mice. Nature 445: 106-10; Ricci-Vitiani L, Lombardi D G, Pilozzi E, Biffoni M, Todaro M, Peschle C et al (2007). Identification and expansion of human colon-cancer-initiating cells. Nature 445: 111-5. The expression of miR-140 in the colon cancer stem cells was over 3-fold higher than that in the control bulk cancer cells. Thus, the colon cancer stem cells may utilize miR-140 to slow down cell proliferation and avoid damage caused by chemotherapy. This may be an important novel mechanism in that tumor stem cells acquire slow proliferative phenotype by certain miRNAs such as miR-140 to avoid damage caused by chemotherapy such as methotrexate.

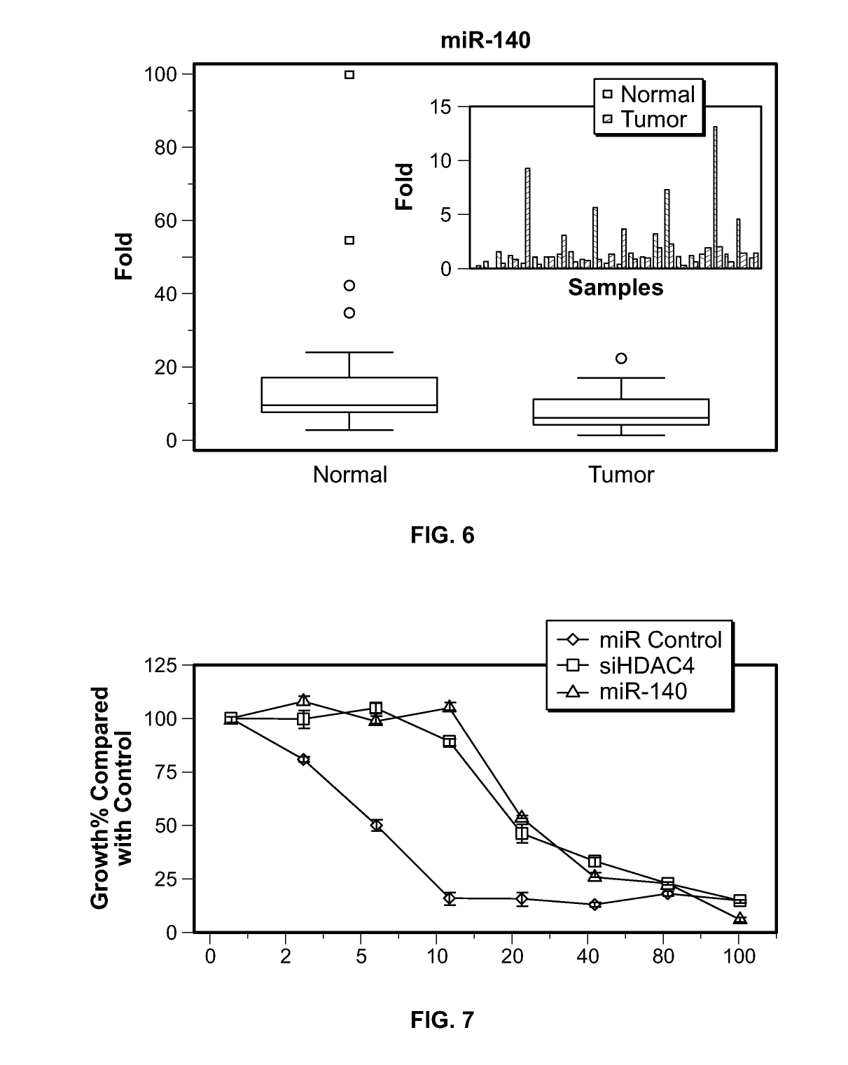

Previous studies have shown that certain miRNAs have close associations with clinical outcomes in colorectal cancer. Nakajima G, Hayashi K, Xi Y, Kudo K, Uchida K, Takasaki K et al (2006). Non-coding MicroRNAs hsa-let-7g and hsa-miR-181b are Associated with Chemoresponse to S-1 in Colon Cancer. Cancer Genomics Proteomics 3: 317-324; and Xi Y, Formentini A, Chien M, Weir D B, Russo J J, Ju J et al (2006). Prognostic Values of microRNAs in Colorectal Cancer. Biomark Insights 2: 113-121. The fact that most of the fast proliferating bulk colon cancer specimens had reduced miR-140 expression levels (FIG. 6) indicates that only a fraction of tumor cells are tumor stem cells with a slow proliferating rate and elevated miR-140, while differentiated tumor cells acquire fast proliferation phenotype by reducing the expression of some of these miRNAs. FIG. 6 shows that the reduction of miR-140 expression levels in tumor specimen compared to expression levels in normal (i.e., non-tumor) tissue varies, but is reduced up to 100 fold.

Previous studies have also shown that several tumor types have high levels of HDAC4. Yang X J, Grégoire S. (2005). Class II histone deacetylases: from sequence to function, regulation, and clinical implication. Mol Cell Biol. 25: 2873-2874. The inventors confirmed that the level of miR-140 was reduced in colorectal tumor specimens which will contribute the elevated levels of HDAC4 (FIG. 6). HDAC4 is also highly expressed in the proliferative compartment in normal colonic and small intestinal epithelium. Wilson (2008). Targeting HDAC4 by histone deacetylase inhibitors may be quite effective for eliminating fast proliferating tumor cells. According to the invention, such inhibitors are made more effective against cancer stem cells that are treated to reduce levels of miR-140.

This disclosure provides a method of increasing proliferation of a cell. In an embodiment of the invention, a cell is contacted with a nucleic acid complementary to at least a portion of miR-140. The amount of nucleic acid complementary to the miRNA is effective to increase proliferation of the cell. In a population of cells, proliferation can determined by observing the proportion of cells in various stages of the cell cycle. For example, according to the invention, contacting cells with miR-140 reduces or prevents arrest in G1 and/or G2. Accordingly, the proportion of cells observed in G1 and/or G2 is reduced. Cell proliferation can also be determined by observing growth rate, for example by measuring optical density or incorporation of labeled nucleotides. In one embodiment, cells that are not cycling are induced to proliferate. In another embodiment, the proliferation rate of a culture or cells increases by at least about 10% or at least about 20% or at least about 50%. The nucleic acid may comprise an antisense nucleic acid, siRNA, shRNA or an anti-miRNA. In certain embodiments, the cell is a cancer stem cell or a neoplastic cell.

In another embodiment, a method of increasing the sensitivity of a cell to a chemotherapeutic agent, is provided. In this embodiment, a cell treated with a chemotherapeutic agent is contacted with a nucleic acid complementary to at least a portion of miR-140. The amount of nucleic acid complementary to the miRNA effective to sensitize the cell to the chemotherapeutic agent is not particularly limited. In one embodiment, the amount is that which induces a cell that is not cycling to proliferate. In another embodiment, that amount is sufficient to increase proliferation in a cell that has not been treated with a chemotherapeutic agent by at least about 10% or at least about 20% or at least about 50%. In another embodiment, the nucleic acid is in an amount that results in increased apoptosis in cells treated with an antineoplastic agent. The increase in apoptosis is at least about 10% or at least about 25%, or at least about 50%, or at least about 100% as compared to a cells treated only with the antineoplastic agent. In certain embodiments, the antineoplastic agent is a chemotherapeutic agent, including, but not limited to, methotrexate, doxorubicin, cisplatin, and ifosfamide. In embodiments, the nucleic acid may comprise and antisense nucleic acid, siRNA, shRNA or an anti-miRNA. In embodiments, the cell may comprise a cancer stem cell or a neoplastic cell.

In another embodiment, a method of increasing the sensitivity of a cell to radiation is provided using the mechanisms of the various pathways disclosed herein. In this embodiment, the cell is contacted with a nucleic acid complementary to at least a portion of miR-140. The amount of nucleic acid complementary to the mRNA is effective to sensitize the cell to radiation and is not particularly limited. In one embodiment, the amount is that which induces a cell that is not cycling to proliferate. In another embodiment, the amount is sufficient to increase proliferation in a cell that has not been treated with a radiation by at least about 10% or at least about 20% or at least about 50%. In another embodiment, the nucleic acid is in an amount that results in increased apoptosis in cells treated with radiation. The increase in apoptosis is at least about 10% or at least about 25%, or at least about 50%, or at least about 100% as compared to cells treated only with radiation. The nucleic acid may comprise and antisense nucleic acid, siRNA, shRNA or an anti-miRNA. In certain embodiments, the cell is a cancer stem cell or a neoplastic cell.

In still another embodiment, the compositions and methods of the present invention involve a first therapy to inhibit miR-140, or expression construct encoding such, used in combination with a second therapy to enhance the effect of the miR-140 therapy, or increase the therapeutic effect of another therapy being employed to treat a neoplasm. These compositions would be provided in a combined amount effective to achieve the desired effect, such as the killing of a cancer cell and/or the inhibition of cellular hyperproliferation. This process may involve contacting the cells with the miR-140 inhibiting or second therapy at the same or different time. This may be achieved by contacting the cell with one or more compositions or pharmacological formulation that includes or more of the agents, or by contacting the cell with two or more distinct compositions or formulations, wherein one composition provides (1) administering to the subject an effective amount of a nucleic acid molecule that inhibits expression of miR-140 and/or (2) a second therapy, in which the inhibition of expression of miR-140 sensitizes the neoplasm to the second therapy.

The second composition or method may comprise administering chemotherapy, radiotherapy, surgical therapy, immunotherapy or gene therapy. For example, in embodiments a chemotherapeutic agent such as, for example, methotrexate, doxorubicin, cisplatin, and ifosfamide is administered. It is contemplated that the combination therapy may be provided in any suitable manner or under any suitable conditions readily apparent to one of ordinary skill in the art.

For example, administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

A wide variety of other chemotherapeutic agents may be used in accordance with the present invention. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

In embodiments, the neoplasm being treated is a form of cancer. Cancers that may be evaluated by methods and compositions of the invention include any suitable cancer cells known to one of ordinary skill in the art. The inventors have found that the present invention is particularly useful in treating cancer cells from the colon or the pancreas, including pancreatic ductal adenocarcinoma. However, other suitable cells include cancer cells of the bladder, blood, bone, bone marrow, brain, breast, cervix, esophagus, gastrointestine, gum, head, kidney, liver, lung, nasopharynx, neck, ovary, prostate, rectum, skin, stomach, testis, tongue, or uterus. Other conditions treatable by the compositions and methods of the present invention will be readily apparent to one of ordinary skill in the art.

An inhibitory nucleic acid molecule of the invention, or other negative regulator of miR-140 may be administered within a pharmaceutically-acceptable diluent, carrier, or excipient, in unit dosage form. Conventional pharmaceutical practice may be employed to provide suitable formulations or compositions to administer the compounds to patients suffering from a neoplasia. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. Therapeutic formulations and methods for making such formulations are well known in the art.

The formulations can be administered to human patients in therapeutically effective amounts to provide therapy for a neoplastic disease or condition. The preferred dosage of inhibitory nucleic acid of the invention is likely to depend on such variables as the type and extent of the disorder, the overall health status of the particular patient, the formulation of the compound excipients, and its route of administration.

Therapy may be provided at any suitable location and under any suitable conditions. The duration of the therapy depends on various factors readily understood by one of ordinary skill in the art. Drug administration may also be performed at any suitable interval. For example, therapy may be given in predetermined on-and-off intervals as appropriate.

Depending on the type of cancer and its stage of development, the therapy can be used to slow the spreading of the cancer, to slow the cancer's growth, to kill or arrest cancer cells, to relieve symptoms caused by the cancer, or to prevent cancer. As described herein, if desired, treatment with an inhibitory nucleic acid molecule of the invention may be combined with therapies such as, for example, radiotherapy, surgery, or chemotherapy for the treatment of proliferative disease.

In another embodiment, a method of diagnosing a neoplasm in a subject is provided. In this embodiment, the method comprises determining the level of expression of at least one of miR-140 and HDAC4.

As described herein, the present invention has identified increases in the expression of miR-140, and corresponding decreases in the expression of HDAC4 that are associated with cellular proliferation. Determining alterations in the expression level of one or more other markers typically used to diagnose a neoplasia are also contemplated. If desired, alterations in the expression of any combination of these markers is used to diagnose or characterize a neoplasia as would be readily apparent to one of ordinary skill in the art.

In an embodiment, a subject is diagnosed as having or having a propensity to develop a neoplasia, the method comprising measuring markers in a biological sample from a patient, and detecting an alteration in the expression of test marker molecules relative to the sequence or expression of a reference molecule. While the following approaches describe diagnostic methods featuring miR-140, the skilled artisan will appreciate that any one or more other markers may also be useful in such diagnostic methods. Expression of a miR-140 is correlated with neoplasia. Accordingly, the invention provides compositions and methods for characterizing a neoplasia in a subject. The present invention provides a number of diagnostic assays that are useful for the identification or characterization of a neoplasia. Alterations in gene expression are detected using methods known to the skilled artisan and described herein. Such information can be used to diagnose a neoplasia.

In an embodiment, diagnostic methods of the invention are used to assay the expression of miR-140 in a biological sample relative to a reference sample. In one embodiment, the level of miR-140 is detected using a nucleic acid probe that specifically binds miR-140. Exemplary nucleic acid probes that specifically bind miR-140 are described herein.

In an embodiment, quantitative PCR methods are used to identify an increase in the expression of miR-140. In another embodiment, PCR methods are used to identify an alteration in the sequence of miR-140. The invention provides probes that are capable of detecting miR-140. Such probes may be used to hybridize to a nucleic acid sequence derived from a patient having a neoplasia. The specificity of the probe determines whether the probe hybridizes to a naturally occurring sequence, allelic variants, or other related sequences. Hybridization techniques may be used to identify mutations indicative of a neoplasia or may be used to monitor expression levels of these genes.

In certain embodiments, a measurement of a nucleic acid molecule in a subject sample may be compared with a diagnostic amount present in a reference, such as a normal control. Any significant increase or decrease in the level of test nucleic acid molecule or polypeptide in the subject sample relative to a reference may be used to diagnose a neoplasia. Test molecules include any one or more of markers disclose herein. In an embodiment, the reference is the level of test polypeptide or nucleic acid molecule present in a control sample obtained from a patient that does not have a neoplasia. In another embodiment, the reference is a baseline level of test molecule present in a non-neoplastic (i.e., normal) sample derived from a patient prior to, during, or after treatment for a neoplasia. In yet another embodiment, the reference can be a standardized curve.

In another embodiment, a method of identifying a neoplasm resistant to chemotherapy is provided. In this embodiment, the method comprises determining the level of expression in the neoplasm of miR-140, and identifying the neoplasm as resistant to therapy if the level of the miR-140 is elevated. As disclosed herein, miR-140 levels in colorectal cancer specimens are reduced compared to paired normal mucosa or other normal tissue (i.e., a normal control). Thus, elevated miR-104 includes a level equivalent to that in normal tissue, as well as a level that is at least 2×, 5×, 10× or higher relative to that in normal tissue. Normal miR-140 levels may be determined over samples from a range of patients. Accordingly, miR-140 levels in a pathological sample can be compared to a base value determined over a range of normal samples rather than for each subject individually.

In another embodiment, a method of determining whether a neoplasm is a candidate for treatment with a chemotherapeutic agent is provided. In one such embodiment, the method comprises evaluating the level of expression of miR-140 and rejecting the candidate if expression of the miR-140 is elevated, or identifying the candidate as suitable for coadministration of chemotherapeutic agent and an agent that promotes miR-140 function and/or cell proliferation. As above, elevated miR-104 includes a level equivalent to that in normal mucosa or other normal tissue, as well as a level that is at least 2×, 5×, 10× or higher relative to the normal tissue.

In another embodiment, a kit for analysis of miR-140 in a pathological sample is provided. Any of the compositions described herein may be comprised in the kit. In a non-limiting example, reagents for isolating miRNA, labeling miRNA, and/or evaluating a miRNA population using an array, nucleic acid amplification, and/or hybridization can be included in a kit, as well reagents for preparation of samples from blood samples. Hybridization probes can include any of the aforementioned natural and synthetic nucleic acids and nucleic acid analogs. The kit may further include reagents for creating or synthesizing miRNA probes. The kits may comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, the kit may comprise, in suitable container means, any suitable solvents, buffers, reagents, or additives known to one of ordinary skill in the art including, but not limited to, those generally used for manipulating RNA, such as formamide, loading dye, ribonuclease inhibitors, and DNase.

In other embodiments, kits may include an array containing miRNA probes. Such arrays may include, for example, arrays relevant to a particular diagnostic, therapeutic, or prognostic application. For example, the array may contain one or more probes that is indicative of a disease or condition, susceptibility or resistance to a drug or treatment, susceptibility to toxicity from a drug or substance, prognosis, and/or genetic predisposition to a disease or condition.

For any kit embodiment, including an array, there can be nucleic acid molecules that contain or can be used to amplify a sequence that is a variant of, identical to or complementary to all or part of any of SEQ IDs described herein. In certain embodiments, a kit or array of the invention can contain one or more probes for the miRNAs identified by the SEQ IDs described herein. Any nucleic acid discussed above may be implemented as part of a kit.

The components of the kits may be packaged in any suitable manner known to one of ordinary skill in the art such as, for example, in aqueous media or in lyophilized form. The kits of the present invention may also include a means for containing the nucleic acids, and any other reagent containers in close confinement for commercial sale. Such containers may include injection or blow molded plastic containers into which the desired vials are retained.

A non-limiting embodiment of a kit described herein may contain reagents to extract RNA from tissue biopsies or cells sorted by FACS (i.e., fluorescence activated cell sorting), reagents to reverse transcribe the isolated RNA into cDNA, reagents to amplify the obtained cDNA and reagents to quantify the amount of amplified DNA obtained. Such reagents may be commercially obtained from Qiagen, Ambion, Clontech, and Stratagene, and similar companies known by the person of ordinary skill in the art. Reagents for extraction of RNA from tissues and cells are known in the art (See e.g., Sambrook, J. and Russel, D. W., (2001) Molecular Cloning: A Laboratory Manual, Third Edition. Cold Spring Harbor Laboratory Press, Cold Spring Harbor, N.Y.; and Current Protocols in Molecular Biology, (2001) John Wiley & Sons, Inc.). Reagents to reverse transcribe isolated RNA into cDNA are also known in the art and include, for example, reverse transcriptase enzyme, an appropriate buffer, random primers or primers specific for the miR-140 sequence (see SEQ ID NO:1) and deoxyribonucleotides. Reagents to amplify the obtained cDNA are also known in the art and include, for example, Taq polymerase, an appropriate buffer, primers specific for miR-140 (see SEQ ID NO:1) and desoxyribonucleotides. Reagents and techniques to quantify an amount of DNA obtained by quantitative PCR amplification are also well known in the art (See e.g., Sambrook, J. and Russel, D. W., (2001) Molecular Cloning: A Laboratory Manual, Third Edition. Cold Spring Harbor Laboratory Press, Cold Spring Harbor, N.Y.; and Current Protocols in Molecular Biology, (2001) John Wiley & Sons, Inc.). A non-limiting example of a reagent that may be used to quantify DNA includes SYBR Green, which is a dye that binds to DNA and fluoresces. SYBR Green may be added to the PCR reaction and the amplified DNA is quantified based on the amount of fluorescence detected. PCR cyclers that can perform such detections include those commercially available from Applied Biosystems.

In such embodiments, the kits may also include components that facilitate isolation of the labeled miRNA. It may also include components that preserve or maintain the miRNA or that protect against its degradation. Such components may be RNAse-free or protect against RNases. Such kits generally will comprise, in suitable means, distinct containers for each individual reagent or solution.

A kit will also include instructions for employing the kit components as well the use of any other reagent not included in the kit. Instructions may include variations that can be implemented.

A method of identifying an agent that inhibits the expression or activity of miR-140 is provided. In one embodiment, the method comprises contacting a cell that expresses the miR-140 with an agent, and comparing the expression level of the miR-140 in the cell contacted by the agent with the expression level of the miR-140 in the absence of the agent. According to this embodiment, the agent is an inhibitor of the miR-140 if expression of the miR-140 is reduced. In this embodiment, the test cell has altered expression of the miRNA, for example, overexpression of miR-140.

Compounds that modulate the expression or activity of a miR-140 nucleic acid molecule, variant, or portion thereof are useful in the methods of the invention for the treatment or prevention of a neoplasm. The method of the invention may measure a decrease in transcription of miR-140 or an alteration in the transcription or translation of the target of miR-140. Any number of methods are available for carrying out screening assays to identify such compounds. In an embodiment, the method comprises contacting a cell that expresses miR-140 with an agent and comparing the level of miR-140 expression in the cell contacted by the agent with the level of expression in a control cell, wherein an agent that decreases the expression of miR-140 thereby, in combination with a secondary therapy, inhibits a neoplasia. In another embodiment, candidate compounds are identified that specifically bind to and alter the activity of miR-140 of the invention. Methods of assaying such biological activities are known in the art. The efficacy of such a candidate compound is dependent upon its ability to interact with miR-140. Such an interaction can be readily assayed using any number of standard binding techniques and functional assays.

Potential agonists and antagonists of miR-140 include, but are not limited to, organic molecules, peptides, peptide mimetics, polypeptides, nucleic acid molecules, and antibodies that bind to a nucleic acid sequence of the invention and thereby inhibit or extinguish its activity. Potential antagonists also include small molecules that bind to miR-140 thereby preventing binding to cellular molecules with which the miRNA normally interacts, such that the normal biological activity of the miRNA is reduced or inhibited. Small molecules of the invention preferably have a molecular weight below 2,000 daltons, more preferably between 300 and 1,000 daltons, and still more preferably between 400 and 700 daltons. It is preferred that these small molecules are organic molecules.

The invention also includes novel compounds identified by the above-described screening assays. These compounds are characterized in one or more appropriate animal models to determine the efficacy of the compound for the treatment of a neoplasia. Characterization in an animal model can also be used to determine the toxicity, side effects, or mechanism of action of treatment with such a compound. Furthermore, novel compounds identified in any of the above-described screening assays may be used for the treatment of a neoplasia in a subject. Such compounds are useful alone or in combination with other conventional therapies known in the art.

It is also contemplated that the invention can be used to evaluate differences between stages of disease, such as between hyperplasia, neoplasia, precancer and cancer, or between a primary tumor and a metastasized tumor. Moreover, it is contemplated that samples that have differences in the activity of certain pathways may also be compared. It is further contemplated that nucleic acids molecules of the invention can be employed in diagnostic and therapeutic methods with respect to any of the above pathways or factors. Thus, in some embodiments of the invention, a miRNA may be differentially expressed with respect to one or more of the above pathways or factors.

In certain embodiments, miRNA profiles may be generated to evaluate and correlate those profiles with pharmacokinetics. For example, miRNA profiles may be created and evaluated for patient tumor and blood samples prior to the patient's being treated or during treatment to determine if there are miRNAs whose expression correlates with the outcome of the patient. Identification of differential miRNAs can lead to a diagnostic assay involving them that can be used to evaluate tumor and/or blood samples to determine what drug regimen the patient should be provided. In addition, it can be used to identify or select patients suitable for a particular clinical trial. If a miRNA profile is determined to be correlated with drug efficacy or drug toxicity, that may be relevant to whether that patient is an appropriate patient for receiving the drug or for a particular dosage of the drug.

In addition to the above prognostic assay, blood samples from patients with a variety of diseases can be evaluated to determine if different diseases can be identified based on blood miRNA levels. A diagnostic assay can be created based on the profiles that doctors can use to identify individuals with a disease or who are at risk to develop a disease. Alternatively, treatments can be designed based on miRNA profiling.

All references mentioned herein are incorporated in their entirety by reference into this application.

It is to be understood and expected that variations in the principles of invention herein disclosed may be made by one skilled in the art and it is intended that such modifications are to be included within the scope of the present invention. The following examples only illustrate particular ways to use the novel technique of the invention, and should not be construed to limit the scope of the invention in any way.

### Examples

The threshold cycle (CT) value for each target was determined by SDS software v1.2 (Applied Biosystems Inc.). Expression levels of each miRNAs were normalized by calculating the ΔCT values based on subtracting the CT value of target miRNA from the CT value of the internal control RNU6B. Sample with the highest expression levels of miRNAs was used as 100% to generate relative expression values. Statistical studies were performed using MedCalc® for Windows, version 8.1.1.0 (MedCalc software, Mariakerke, Belgium). Statistical differences of the expression level between tumor and normal tissues for each target were calculated by Wilcoxon test. Statistical significance was set as a p<0.05.

Translational Regulation of HDAC4 Expression by miR-140

Cells were plated in six-well plates at a density of 2×10⁵ cells/well. The next day, cells were transfected with 100 nM of miR-140 precursor or non specific miR control (Ambion, Inc.) with Oligofectamine (Invitrogen Inc.) based on the manufacturer's instructions. Positive control siRNA specific against HDAC4 (ON-TARGET plus SMARTpool L-003497-00-0010, human HDAC4, NM 006037) was purchased from Dharmacon and transfected with Oligofectamine according to the manufacturer's protocols at a final concentration of 100 nM.

Total RNA, including miRNAs, was isolated from cell lines or clinical specimens by using TRIzol reagent (Invitrogen, Inc.) according to the manufacturer's instructions to determine whether the cells were transfected with miR control, miR-140 or siHDAC4 at a final concentration of 100 nM for 24 hrs before RNA isolation.

The concentration of isolated RNAs was determined by Nanodrop and the integrity of the RNAs was analyzed by RNA bioanalyzer (Bio-Rad, Inc). cDNA synthesis was carried out with the High Capacity cDNA synthesis kit (Applied Biosystems) using 5 ng of total RNA as template. The miRNA sequence-specific RT-PCR primers for miR-140 and endogenous control RNU6B were purchased from Ambion. Real-time quantitative PCR analysis was carried out using Applied Biosystems 7500 Real-Time PCR System. The PCR master mix containing TaqMan 2× Universal PCR Master Mix (No Amperase UNG), 10× TaqMan assay and RT products in 20 ul volume were processed as follows: 95° C. for 10 min, and then 95° C. for 15 sec, 60° C. for 60 sec for up to 40 cycles (n=3). Signal was collected at the endpoint of every cycle. The gene expression CT values of miRNAs from each sample were calculated by normalizing with internal control RNU6B and relative quantitation values were plotted.

cDNA was synthesized with the High Capacity cDNA synthesis kit (Applied Biosystems) using 2 μg of total RNA as the template and 10× random primers. Real-time qPCR analysis was done on the experimental mRNAs. The PCR primers and probes for HDAC4, and the internal control gene GAPDH were purchased from Applied Biosystems. qRT-PCR was done on an ABI 7500HT instrument under the following conditions: 50° C., 2 min for one cycle; 95° C., 10 min; 95° C., 15 s; 60° C., 1 min for 40 cycles (n=3).

Cells were plated in 96-well plates with 6 repeats at 2,000 cells/well after transfection with miR-140 or miR control. Cells were cultured for 24, 48, 72, 96 and 120 h. The absorbance at 450 and 630 nm was measured after incubation with 10 μl of WST-1 for 1 h.

Cells were transfected with miR-140 and miR control described as above. At 36 h after transfection, cells were harvested and resuspended at 0.5-1×10⁵ cells/ml in modified Krishan buffer (He, 2007; Tarasov, 2007), containing 0.1% sodium citrate and 0.3% NP-40 and kept at 4° C. Before being analyzed by flow cytometry, cells were treated with 0.02 mg/ml RNase H and stained with 0.05 mg/ml propidium iodide (Sigma).

Forty-eight hours after transfection with miR-140 or miR control, cells were harvested and lysed in 1×RIPA buffer (Sigma) supplied with 100 uM PMSF (sigma) and proteinase inhibitor cocktail (Sigma). Equal amounts of protein were resolved by a 8% SDS-PAGE gels using the method of Laemmli (Laemmli, 1970), and transferred to polyvinylidene fluoride membranes (BIO-RAD Laboratories). The membranes were then blocked by 5% nonfat milk in TBST (Tris-buffered saline and 1% Tween-20) at room temperature for 1 h. The primary antibodies used for the analysis included goat anti-HDAC4 polyclonal Ab (1:1000, N-18), mouse anti-p53 mAb (1:1000, DO-1), mouse anti-p21 mAb(1:1000, F-5), mouse anti-tubulin mAb (1:1000, TU-02), all from Santa Cruz Biotechnology. Horseradish peroxidase-conjugated antibodies against mouse or goat (1:1000, Santa Cruz Biotechnology) were used as the secondary antibodies. Protein bands were visualized with a chemiluminescence detection system using Super Signal substrate (Pierce).

HCT 116 (wt-p53) cells were sorted with multiparametric flow cytometry with BD FACS Aria cell sorter (Becton Dickinson, CA) at sterile conditions. Cells were prepared as described above and labeled with one or several markers conjugated anti-human CD133-PE (clone 105902; R&D Systems, MN); CD44-FITC (clone F10-44-2, R&D Systems, MN). Antibodies were diluted in buffer containing 5% BSA, 1 mM EDTA and 15-20% blocking reagent (Miltenyi Biotec, CA) to inhibit unspecific binding to non-target cells. After 15 min incubation at 4° C., stained cells were washed, resuspended in 500 μl of MACS buffer and sorted.

U-2 OS and HCT 116 (wt-p53) cells were replated in 96-well plates at 2×10³ cells/well in triplicate after transfected with miR-140, miR control, or siRNA against HDAC4 in 100 μl of medium. Twenty-four hours later, methotrexate (ranged from 10-1000 nM) was added and incubated for 72 h. Ten μl of WST-1 (Roche Applied Science) was added to each well. After 1 h incubation, absorbance was measured at 450 and 630 nm respectively. Non-specific miRNA was used as the negative control.

HCT 116 (wt-p53) cells were replated in 96-well plates at 2×10³ cells per well in triplicate after transfected with miR-140, miR control or siRNA against HDAC4 in 100 μl of medium. After 24 h, 5-FU (ranged from 2 to 100 μM) was added and incubated for 72 h. WST-1 (10 μl) was added to each well. After 1 h incubation, absorbance was measured. Nonspecific miR was used as the negative control.

Colon cancer stem-like cells were transfected with 100 nM of LNA anti-miR-140 using Lipofectamine 2000 after FACS-sorting. After 24 h, cells were washed by phosphate buffered saline (PBS) and then incubated with lethal dose of 5-FU (100 mM) for 48 h. The dead cells were determined by the fluorescein isothiocyanate (FITC) Annexin V and PI detection kit (BD Biosciences, Pharmingen, San Diego, Calif., USA). Briefly, cells were harvested and resuspended in 1× Annexin V binding and stained with Annexin V (5 μl) and PI (5 μl) for 15 min at room temperature in the dark. After additional 400 μl of binding buffer, cells were analyzed by flow cytometry. For the sensitivity of 5-FU in the colon cancer stem-like cells and control bulk cancer cells, cells were incubated with 100 mM of 5-FU for 48 h before flow cytometry analysis.

Based on Targetscan analysis for potential miR-140 targets, the seed sequence (5′-GUGGUUU-3′) of both hsa-miR-140 and mmu-miR-140 matches with the potential binding site at the 3′-UTR of HDAC4 mRNA (Lewis et al., 2005; Lewis et al., 2003) (FIG. 1 A). To experimentally confirm that the expression of HDAC4 is indeed regulated by miR-140, we overexpressed miR-140 by transient transfection in U-2 OS (wt-p53) and HCT 116 (wt-p53). A non-specific miR was used as a negative control. Over-expression of miR-140 in four cell lines (FIG. 1 B) was confirmed by real time qRT-PCR analysis using U6 RNA to normalize the expression. We analyzed the expression level of HDAC4 mRNA using real time qRT-PCR analysis in U-2 OS (wt-p53) and HCT 116 (wt-p53) cells. The decreased protein level of HDAC4 by siRNA was clearly caused by mRNA degradation. By contrast, there was no change in HDAC4 mRNA expression by miR-140 treatment (FIG. 1 C, lane 4). The expression of HDAC4 protein was analyzed using Western immunoblot analysis and the results are shown in FIG. 1D. Over-expression of miR-140 clearly decreased the expression of HDAC4 protein without mRNA degradation (FIG. 1D, lane 3). To further confirm that the expression of HDAC4 is regulated by miR-140, loss-of-function analysis was performed by knocking down the endogenous miR-140 with LNA-modified anti-miR-140 in HCT 116 (wtp53) and HCT 116 (null-p53) cells. Scramble-miR (LNA-control) was used as the negative control. The results showed that knocking down endogenous miR-140 by LNA anti-miR-140 can restore the expression of HDAC4 (FIG. 9).

To knock down miR-140, HCT 116 (wt-p53) and HCT 116 (null-p53) cells were transfected with 100 nM of scramble-miR or LNA anti-miR-140 oligonucleotides (Exiqon, Woburn, Mass., USA) in the six-well plates (2×10⁵ cells per well) by Lipofectamine 2000 (Invitrogen). Cells were harvested at 72 h after transfection and cellular proteins were extracted. HDAC4 protein was detected by western immunoblot analysis.

Effect of miR-140 on Cellular Proliferation

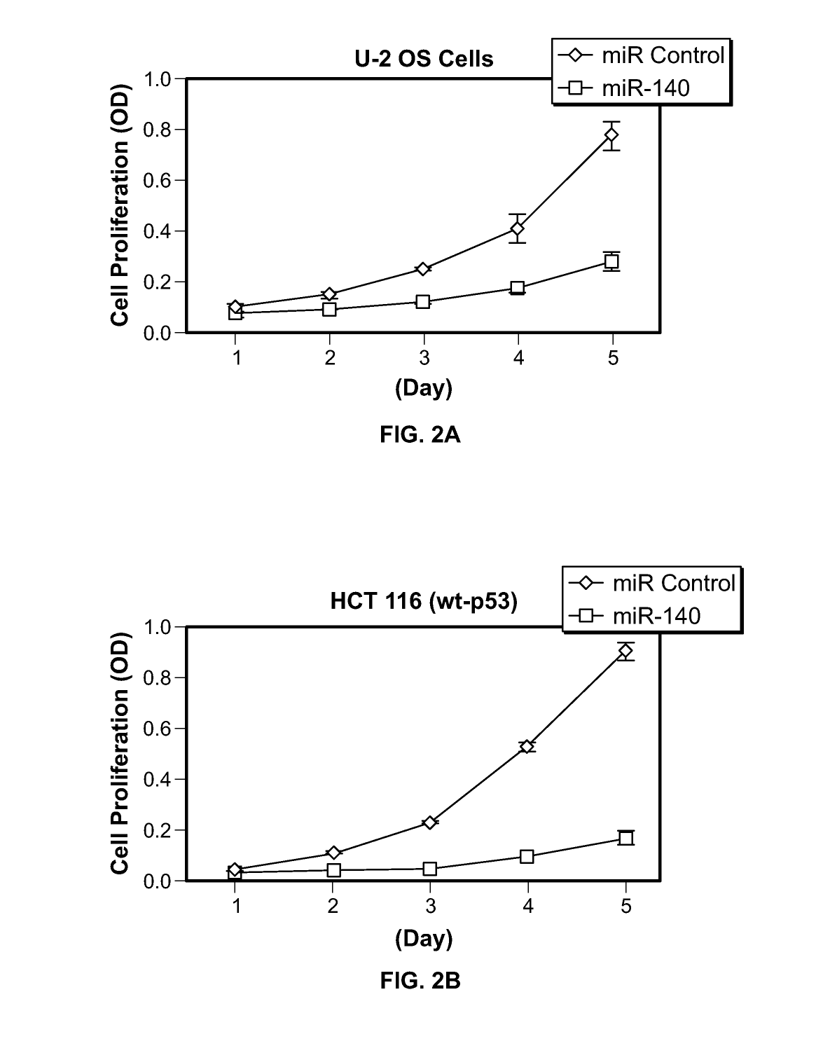

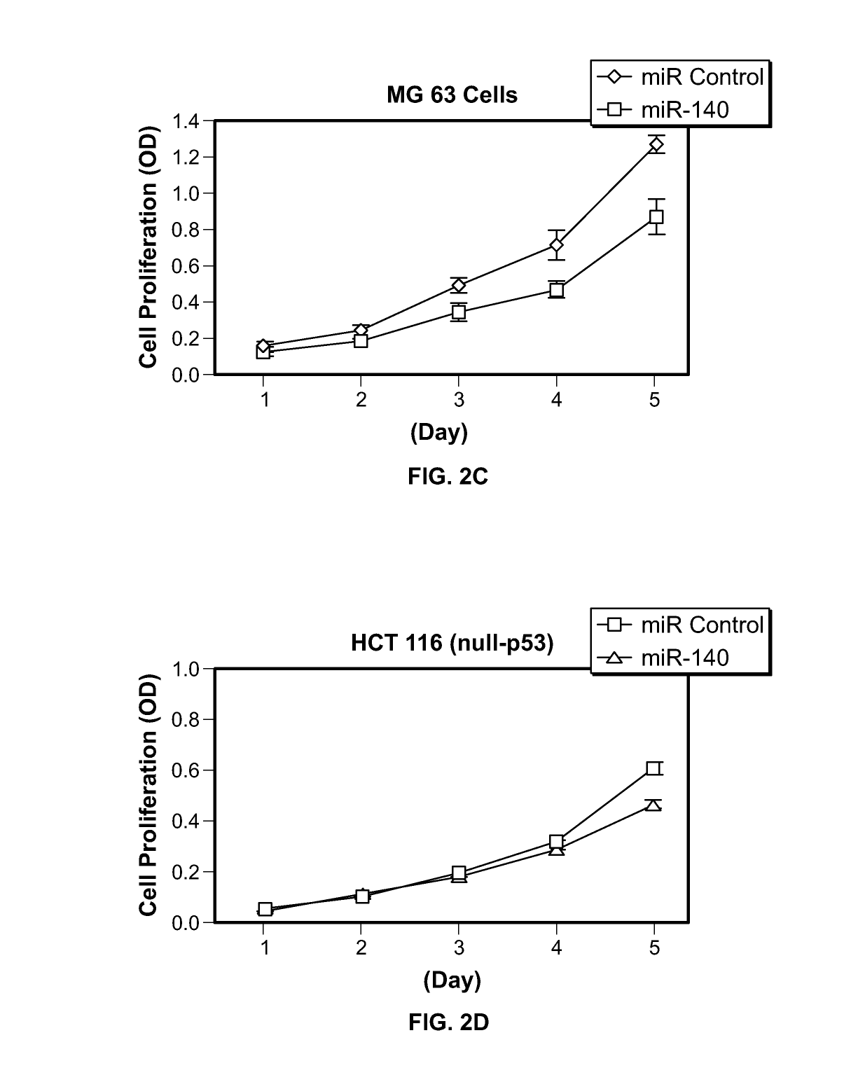

To assess the functional significance of miR-140, we evaluated the impact of miR-140 on cellular proliferation using U-2 OS (wt-p53) cells, MG63 (mut-p53) osteosarcoma cell lines, colon cancer cell lines HCT 116 (wt-p53) and HCT 116 (null-p53). A non-specific miR was used as a negative control. Our results show that the overexpression of miR-140 can suppress cellular proliferation in U-2 OS cells (wt-p53) by 64.05±4.01% (n=6) (FIG. 2A), in HCT 116 (wt-p53) by 81.4±3.75% (n=6) (FIG. 2B), with less impact on MG63 cells (31.3±4.96%, n=6) (FIG. 2C) and HCT 116 (null-p53) cells (22.42±1.88%, n=6) (FIG. 2D) on day 5. By contrast, the miR control has no effect on cellular proliferation (data not show), indicating that this effect caused by miR-140 is highly specific.

Impact of Cell Cycle Control by miR-140

To determine whether the impact of miR-140 on cellular proliferation are related to cell cycle regulation, the effect of miR-140 on cell cycle was analyzed by flow cytometry using U-2 OS cells (wt-p53), MG63 cells (mut-p53), HCT 116 (wt-p53) and HCT 116 (null-p53) cells transfected with miR control or miR-140. miR-140 induces G1 (1.76 fold) but not G2 arrest (0.92 fold) in U-2 OS (wt-p53) cells (FIG. 3A); miR-140 induces both G1 (3.33 fold) and G2 arrest (2.54 fold) in HCT 116 (wt-p53) cells (FIG. 3B). By contrast, this effect has not been observed in MG63 cells (mut-p53) or HCT 116 (null-p53) (FIG. 3).

Effect of miR-140 on Cell Cycle Regulating Genes

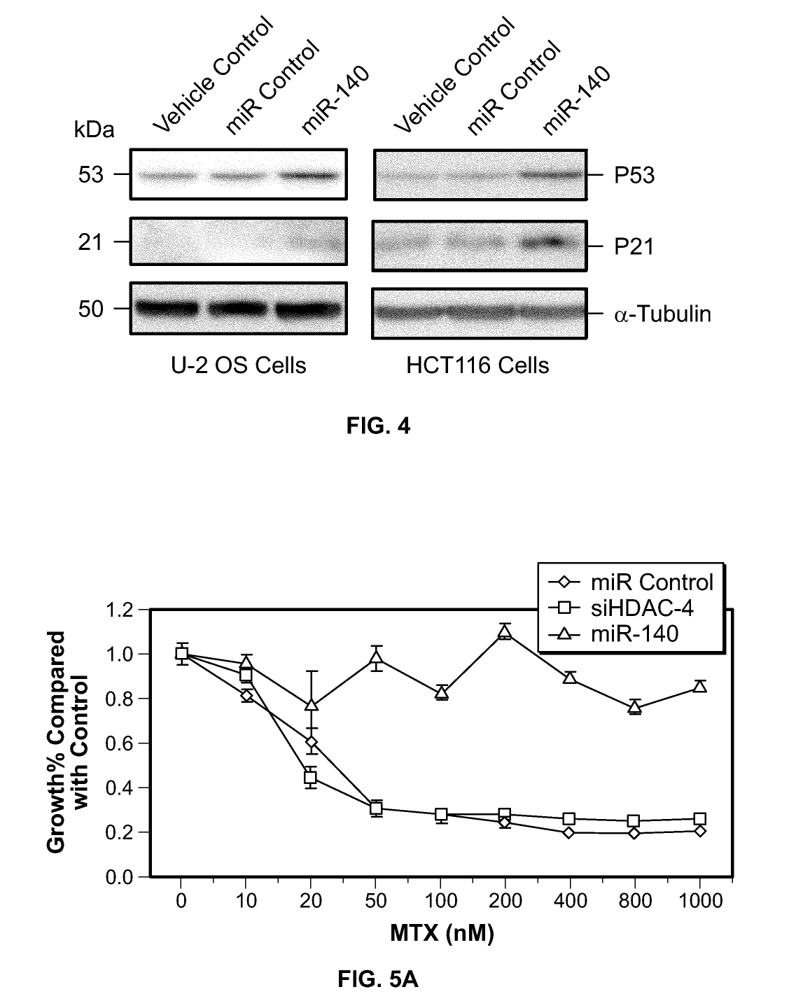

To further analyze the cell cycle regulating genes relating to miR-140 overexpression, the cell cycle regulating genes p53 and p21 were observed. FIG. 4 shows the results of p53 and p21 expression determined by Western immunoblot analysis in U-2 OS (wt-p53) cells and in HCT 116 (wt-p53) (FIG. 4). Ectopic overexpression of miR-140 increased the expression of both p53 and p21 proteins (FIG. 4, lane 3).

Over-Expression of miR-140 Causes Reduced Chemosensitivity to Methotrexate

The effect of miR-140 on chemosensitivity to methotrexate treatment was characterized. HCT116 (wt-p53) cells was transfected with miR-140, miR control, and siRNA against HDAC4 to evaluate the impact of miR-140 on chemosensitivity. Cells with elevated miR-140 were more resistant to methotrexate compared to miR control (FIG. 5A).

Over-Expression of miR-140 Causes Reduced Chemosensitivity to 5-Fluorouracil

The effect of miR-140 on chemosensitivity to 5-fluorouracil treatment was characterized. HCT116 (wt-p53) cells were transfected with miR-140, miR control, and siRNA against HDAC4 to evaluate the impact of miR-140 on chemosensitivity. Cells transfected with miR-140 and those transfected with siRNA against HDAC4 were more resistant to 5-fluorouracil compared to miR control (FIG. 7).

Elevated Expression of miR-140 in Human Colon Cancer Stem Cells May Contribute to Chemoresistance

To determine that colon cancer stem cells may have higher levels of miR-140 expression to process slow proliferating phenotype thereby avoiding damage caused by chemotherapeutic agents, the colon cancer stem cells were isolated using both CD133 and CD44 as selection marker from HCT 116 (wt-p53) cells. The expression of miR-140 in colon cancer stem cells was found to be nearly 4-fold higher than that in the control bulk cancer cells (FIG. 5B, C). The results suggest that colon cancer stem cells may utilize miR-140 to slow down cell proliferation and avoid damage caused by chemotherapy until receiving a proliferation and differentiation signal, further verifying the impact of miR-140 on cell proliferation and chemotherapy resistance.

CD133^{+hi}CD44^{+hi} Colon Cancer Stem-Like Cells are More Resistant to 5-Fluorouracil (5-FU) Treatment.

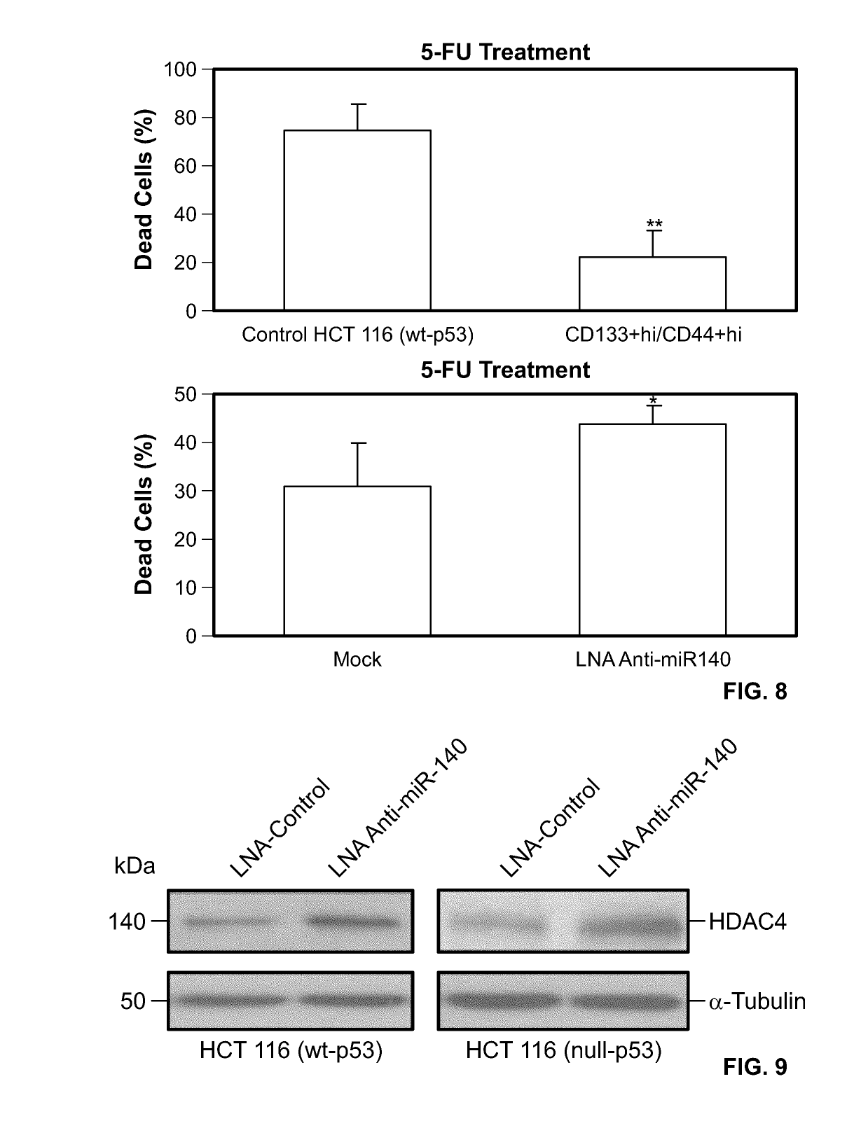

FACS-sorted CD133^{+hi}/CD44^{+hi} colon cancer stem-like cells were far more resistant (about 20% cell death) to high-dose 5-FU treatment than nonsorted control HCT 116 (wt-p53) cells (>80% cell death) (FIG. 8, top). To directly demonstrate that the chemoresistance to 5-FU treatment in CD133^{+hi}/CD44^{+hi} cells can be reversed, the expression of miR-140 was knocked down by LNA-modified anti-miR-140. The results showed that CD133^{+hi}CD44^{+hi} cells with reduced level of miR-140 by LNA-anti-miR-140 were more sensitive to 5-FU treatment compared to LNA-control treated cells (FIG. 8, bottom).

Expression of miR-140 was Decreased in Colorectal Cancer Specimens

To evaluate miR-140 expression level in colon cancer patients, miR-140 levels in 24 fresh frozen colorectal cancer specimens were compared with their paired adjacent normal specimens using real time qRT-PCR analysis. The results showed that the expression levels of miR-140 were significantly reduced compared to normal tissues (p<0.05) (FIG. 6).

Total RNA, including miRNAs, was isolated from cell lines, tumor xenografts or clinical specimens using TRIzol reagent (Invitrogen) according to the manufacturer's instructions. cDNA synthesis was carried out with the High Capacity cDNA synthesis kit (Applied Biosystems, Branchburg, N.J., USA) using 5 ng of total RNA as template. The miRNA sequence-specific reverse transcription (RT)-PCR primers for miR-140 and endogenous control RNU6B were purchased. (Ambion; Eurogentec). Real-time-PCR analysis was carried out using Applied Biosystems 7500 Real-Time PCR System (for details, see Song et al., 2008). The gene expression threshold cycle (CT) values of miRNAs from each sample were calculated by normalizing with internal control RNU6B and relative quantitation values were plotted.

What is claimed is:

1. A method of diagnosing whether a neoplasm is resistant to chemotherapy comprising (i) determining the level of at least one of miR-140 and HDAC4 in the neoplasm; (ii) comparing the level of miR-140 and/or HDAC4 in the neoplasm to the level in a normal control; and (iii) identifying the neoplasm as chemotherapy resistant if the level of miR-140 is greater in the neoplasm and/or the level of HDAC4 is less in the neoplasm than in the normal control.

2. The method of claim 1, wherein the level of miR-140 in the neoplasm is determined.

3. The method of claim 1, wherein the level of HDAC4 in the neoplasm is determined.

4. The method of claim 1, further comprising the step of (iv) rejecting the neoplasm as a candidate for treatment with chemotherapy if the level of miR-140 is greater than, or the level of HDAC4 is less than, in the normal control.

5. The method of claim 4, wherein chemotherapy is rejected if the level of miR-140 in the neoplasm is more that 2× the level in normal tissue.

6. The method of claim 4, wherein chemotherapy is rejected if the level of miR-140 in the neoplasm is more that 5× the level in normal tissue.

7. The method of claim 4, wherein the chemotherapy is selected from methotrexate, doxorubicin, cisplatin, and ifosfamide.

8. The method of claim 1, wherein the normal control is selected from the group consisting of a non-neoplastic sample and a standard curve derived from non-neoplastic samples.

9. A method of diagnosing whether a neoplasm comprises a subpopulation of cells resistant to chemotherapy comprising (i) isolating the subpopulation of cells; (ii) determining the level of at least one of miR-140 and HDAC4 in the subpopulation of cells; (iii) comparing the level of miR-140 and/or HDAC4 in the subpopulation of cells to the level in a control sample; and (iv) identifying the subpopulation of cells as chemotherapy resistant if the level of miR-140 is greater in the subpopulation, and/or the level of HDAC4 is less in the subpopulation, than in the control sample.

10. The method of claim 9, wherein the control sample is bulk cancer cells.

11. The method of claim 9, wherein the control sample is normal tissue.

12. The method of claim 9, wherein the subpopulation of cells are cancer stem cells.
